(19) United States
(12) Patent Application Publication
(10) Pub. No.: US 2006/0090215 A1
(43) Pub. Date: Apr. 27, 2006

(54) Low allergen plant and animal genotypes

(76) Inventors: Bob B. Buchanan, Oakland, CA (US); Peggy Lemaux, Moraga Town, CA (US); Joshua H Wong, San Francisco, CA (US); Hyun-Kyung Kim, San Francisco, CA (US); Myeong-Le Cho, Alameda, CA (US); Oscar L Frick, San Francisco, CA (US)

Correspondence Address: MORRISON & FOERSTER LLP, 425 MARKET STREET, SAN FRANCISCO, CA, 94105-2482, US

(21) Appl. No.: 10/510,325

(22) PCT Filed: Apr. 11, 2003
(86) PCT No.: PCT/US03/10910

§ 371 Date: Aug. 22, 2005

Related U.S. Application Data
(60) Provisional application No. 60/372,253, filed on Apr. 11, 2002.

(51) Int. Cl. A01H 1/00; C12Q 1/68; C12N 5/04; C12N 15/82

(52) U.S. Cl. 800/278; 435/6; 435/419; 435/468

(57) ABSTRACT

The selection of low allergen plant and animal subspecies, subgroups, and genotypes for production of low allergen food products is described.

### RELATED APPLICATION

This application claims the benefit of U.S. Provisional Application 60/372,253, filed Apr. 11, 2002, the disclosure of which is hereby incorporated by reference.

### FIELD OF THE INVENTION

This invention is in the field of food allergens. In particular, this invention relates to the selection of low allergen plant and animal genotypes for production of low allergen food products. In addition, this invention relates to reduced allergen plants and animals and low allergen food products produced therefrom.

### BACKGROUND OF THE INVENTION

A food allergy, or hypersensitivity, is an abnormal response to a food triggered by the immune system. About 1.5 percent of adults and up to 6 percent of children younger than 3 years in the United States—about 4 million people—have a food allergy.

It is critical for people who have food allergies to identify them and to avoid foods that cause allergic reactions. Some foods can cause severe illness and, in some cases, a life-threatening allergic reaction (anaphylaxis) that can constrict airways in the lungs, severely lower blood pressure, and cause suffocation by the swelling of the tongue or throat.

While it is important for people with food allergies to avoid foods that cause allergic reactions, it is not always possible. Some food allergens are carried in the air making it extremely difficult for an allergic person to avoid all exposures to the allergens. As such, there is a tremendous need to identify reduced allergen food components to produce reduced allergen food products.

### SUMMARY OF THE INVENTION

In order to meet these needs, the present invention is directed to a method for selecting a genotype within a species that induces a reduced allergic reaction in an allergy test compared to other genotypes within the species. The method of the invention generally includes the following steps: (a) isolating protein fractions from the genotypes; (b) testing the protein fractions for an allergic reaction in an allergy test and (c) selecting a genotype within the species that exhibits a reduced allergic reaction compared to other genotypes within the species in the allergy test. However, some allergens may be sufficiently allergenic that step (a) is unnecessary and the allergen itself may be tested directly in step (b) rather than protein fractions. In the method, the species may be a plant or animal species. Plant species may include but are not limited to wheat, barley, corn, rice, soybean, peanut, Brazil nut, English walnut, kiwis. Animal species may include but are not limited to cows, chickens, shellfish and fish. One of skill in the art will recognize that in addition to selecting particular genotypes that exhibit a reduced allergic reaction, the methods of the present invention may also be used to select subspecies and other subgroups that exhibit low allergic reaction. Furthermore, the methods of the present invention may be used to identify previously unrecognized subgroups based upon their low allergic reaction compared to the bulk population of a given species.

The invention is also directed to methods of identifying growth conditions that lead to a reduced allergenic reaction in an allergy test compared to the same species, subspecies, subgroup, or preferably a genotype grown under different conditions. The method of the invention generally includes the following steps: (a) growing a species under a variety of different conditions; (b) isolating protein fractions from the species from each growth condition; (c) testing the protein fractions for an allergic reaction in an allergy test and (d) selecting a growth condition that exhibits a reduced allergic reaction compared to other growth conditions in the allergy test. However, some allergens may be sufficiently allergenic that step (a) is unnecessary and the allergen itself may be tested directly in step (b) rather than protein fractions.

The invention is further directed to food and food products produced from low allergic reaction-inducing genotypes. Some low allergic reaction-inducing genotypes may be consumed directly: low allergen peanuts, chickens, shellfish, nuts, rice, milk from cows, etc. Other, low allergic reaction-inducing genotypes may be combined into food products for consumption. For example, low allergen wheat can produce low allergen bread. Low allergen cows can produce low allergen milk for use in cakes, etc. Furthermore, low allergen yeast may be used to limit allergic reactions to airborne yeast when preparing foods with the yeast and to produce low allergen foods.

The method of the invention may further include the additional step of genetically modifying the selected genotype to further reduce the allergic reaction inducing response of the genotype. The selected genotype may be genetically modified by traditional breeding methods (selecting, crossing) and/or by genetic engineering techniques.

The invention is further directed to transgenic plants and animals and products produced therefrom wherein the plants and animals overexpress proteins involved in the pentose phosphate pathway to make the plants and animals less allergenic. Such proteins include thioredoxin, NTR and glucose 6-phosphate dehydrogenase and homologues thereof.

In plants, overexpression of proteins involved in the pentose phosphate pathway in seed effect a significant increase in the reduction of proteins of the albumin fraction (SH as compared to S-S) of the seed. In particular, this invention is directed to transgenic plants that overexpress thioredoxin, NTR and/or glucose 6-phosphate dehydrogenase in various combinations wherein the overexpression of these proteins effects a significant change in the redox state of members of the alpha-amylase inhibitor, the alpha-amylase/trypsin inhibitor and/or the sulfur-rich gliadin families of the seed. As a result, the plant products of the invention are less allergenic than non-transgenic counterpart products. As such, the invention is further directed to hypoallergenic plant products produced from the transgenic plants of the invention.

In one format, transgenic wheat and wheat products are produced by the methods of the invention. Wheat products produced from the transgenic wheat of the invention include reduced alpha-amylase/trypsin inhibitors and exhibit a decreased ability to inhibit trypsin and an increased susceptibility to heat and digestion by trypsin. As a result, the wheat products of the invention are more digestible than non-transgenic counterpart wheat products. As such, the invention is directed to hyperdigestible wheat products produced from the transgenic wheat of the invention.

The invention is further directed to transgenic wheat grain harvested from the transgenic wheat plants of the invention. The invention is further directed to transgenic wheat flour produced from the transgenic wheat grain of the invention. The transgenic wheat flour exhibits reduced Baker's asthma inducing qualities. Furthermore, the invention is directed to wheat food products produced from the transgenic wheat flour of the invention. The wheat food products produced from the transgenic wheat flour of the invention are less allergenic and more digestible than non-transgenic counterparts.

The invention is further directed to a method of producing transgenic wheat flour with reduced baker's asthma-inducing qualities, including (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat and (e) preparing wheat flour from the harvested wheat wherein the wheat has reduced Baker's asthma-inducing qualities.

The invention is further directed to a method of producing transgenic wheat products with reduced wheat allergy inducing qualities, comprising (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat and (e) preparing wheat products from the harvested wheat wherein the wheat products have reduced wheat allergy inducing qualities.

The invention is further directed to a method of producing transgenic wheat products with an increased ease of gastrointestinal processing for sufferers of coeliac disease, comprising (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat and (e) preparing wheat products from the harvested wheat wherein the wheat products have increased ease of gastrointestinal processing for sufferers of coeliac disease.

In the methods of the invention, the wheat flour comprises proteins in the albumin fraction wherein the proteins exhibit a significant increase (about 11%) in the reduction of proteins in the albumin protein fraction as compared to non-transgenic wheat.

The invention is further directed to a method of producing wheat grain from a transgenic wheat plant with a significant increase in the reduction of proteins in the albumin protein fraction of the wheat grain, comprising (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat wherein the wheat grain has a significant increase in the reduction of proteins in the albumin protein fraction of the wheat grain as compared to a non-transgenic wheat plant.

The invention is further directed to a method of producing wheat grain from a transgenic wheat plant with a decrease (10-20% or more) in the abundance of members of the alpha-amylase inhibitor, the alpha-amylase/trypsin inhibitor and/or the sulfur-rich gliadin protein families comprising (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat; wherein the wheat grain has a decrease (10-20% or more) in the abundance of members of alpha-amylase inhibitor, the alpha-amylase/trypsin inhibitor and/or the sulfur-rich gliadin families as compared to a nontransgenic wheat plant.

The invention is further directed to a method of producing wheat grain from a transgenic wheat plant with an altered protein distribution pattern in the albumin fraction, comprising (a) transforming a wheat cell to contain a heterologous DNA segment encoding thioredoxin h wherein the thioredoxin h is operably linked to a promoter for expression of the thioredoxin h in the wheat cell; (b) growing and maintaining the wheat cell under conditions whereby a transgenic wheat plant is regenerated therefrom; (c) growing the transgenic plant under conditions whereby the DNA is expressed and the total amount of thioredoxin h in the plant is increased; (d) harvesting the wheat wherein the wheat grain has an altered protein distribution pattern in the albumin fraction compared to a nontransgenic wheat plant. Illustrative but not limiting of the differences in protein pattern are the differences shown in FIG. 4.

The invention is further directed to a transgenic wheat plant comprising overexpressed thioredoxin h wherein the thioredoxin h is overexpressed in the wheat endosperm resulting in a change in the distribution of proteins in the albumin fraction such that the level of those in the 3.5 to 16 kDa region, including the alpha-amylase and alpha-amylase/trypsin inhibitors is decreased by 10-20% or more in the homozygote vs. the null segregant.

The invention is further directed toward transgenic wheat comprising one or more of the following peptides DCCQQLADISEWCR (SEQ ID NO: 1); EYVAQQTCGVGIVGS (SEQ ID NO: 2); DALLQQCSPVADMSFLR (SEQ ID NO: 3) and SGPWMCYPGQAFQVPALPACR (SEQ ID NO: 4) wherein these peptides are more reduced in the transgenic wheat (SH as compared to S-S) when examined by two dimensional IEF/SDS-PAGE as compared to non-transgenic wheat plants.

### BRIEF DESCRIPTION OF THE DRAWINGS

FIG. 1 shows an elution profile of albumin fraction of transgenic wheat on reversed-phase HPLC.

FIG. 2 shows a one-dimensional SDS/PAGE gel of reversed phase albumin fractions from transgenic wheat with NADPH and NTR.

FIG. 3 shows a scan profile of protein fractions 26 and 28 from reversed phase HPLC C4 column after separation by SDS-PAGE.

FIG. 4 shows a one dimensional SDS-PAGE gel of reversed phase albumin fractions from transgenic wheat without NADPH and NTR.

FIG. 5 shows an isoelectric focussing gel (IEF) for pH 5-8/Tris-Tricine (16.5%) PAGE of albumin fraction from transgenic wheat overexpressing thioredoxin h.

FIG. 6 shows an Alignment of NADP-Thioredoxin Reductases (NTRs) from different sources. Conserved regions in the sequences of the three plants are highlighted. a: Barley (SEQ ID NO: 5); b: Wheat (SEQ ID NO: 6); c: Arabidopsis (SEQ ID NO: 7); d: E. coli. (SEQ ID NO: 8)

FIG. 7 shows an alignment of G6PDHs from different sources. Conserved regions in the sequences of the five plants are highlighted. a: Barley (SEQ ID NO: 9); b: Wheat (SEQ ID NO: 10); c: Rice (SEQ ID NO: 11); d: Tobacco (SEQ ID NO: 12); e: Arabidopsis(SEQ ID NO: 13).

FIG. 8 shows an alignment of thioredoxins from different sources. Conserved regions in the sequences of the five plants are highlighted. a: Barley (SEQ ID NO: 14); b: Wheat (SEQ ID NO: 15); c: Rice (SEQ ID NO: 16); d: Tobacco (SEQ ID NO: 17); e: Arabidopsis (SEQ ID NO: 18); f: E. coli (SEQ ID NO: 19).

FIG. 9 shows the DNA sequence of G6PDH from Barley (SEQ ID NO: 20).

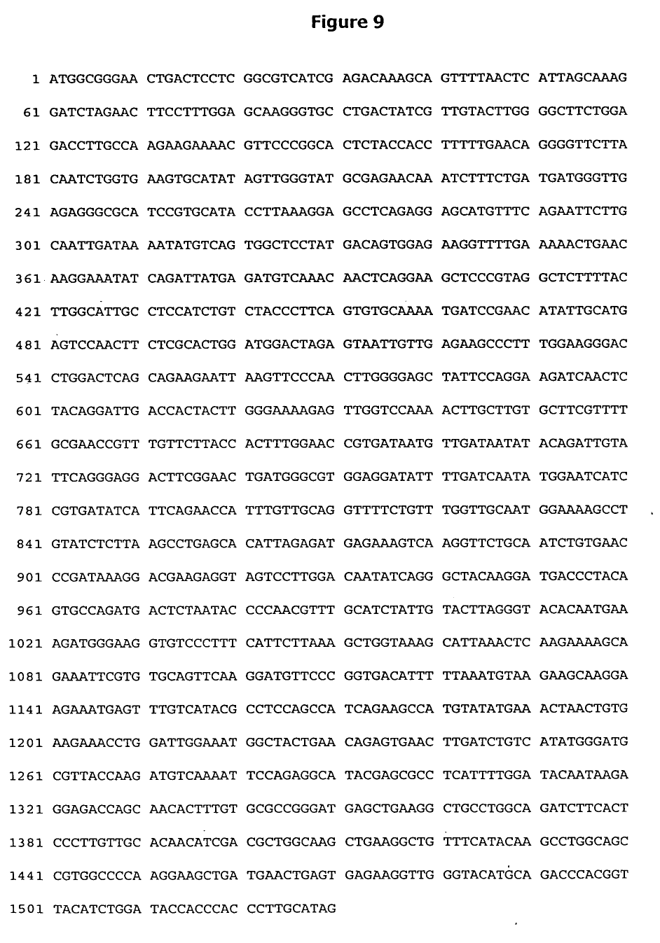

### DETAILED DESCRIPTION OF THE INVENTION

Definitions

The practice of the present invention will employ, unless otherwise indicated, conventional techniques of immunology, molecular biology, plant and animal breeding, microbiology, cell biology and recombinant DNA, which are within the skill of the art. See, e.g., Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987); Plant Breeding: Theory and Techniques S. K. Gupta, Editor. Jodhpur, Agrobios, 2000, 388; Coligan, Dunn, Ploegh, Speicher and Wingfeld, eds. (1995) CURRENT PROTOCOLS IN PROTEIN SCIENCE (John Wiley & Sons, Inc.); the series METHODS IN ENZYMOLOGY (Academic Press, Inc.): PCR 2: A PRACTICAL APPROACH (M. J. MacPherson, B. D. Hames and G. R. Taylor eds. (1995), Harlow and Lane, eds. (1988) ANTIBODIES, A LABORATORY MANUAL, and ANIMAL CELL CULTURE R. I. Freshney, ed. (1987).

Unless otherwise noted, technical terms are used according to conventional usage. Definitions of common terms in molecular biology may be found in Lewin, Genes V, published by Oxford University Press, 1994 (SBN 0-19-854287-9); Kendrew et al. (eds.), The Encyclopedia of Molecular Biology, published by Blackwell Science Ltd., 1994 (SBN 0-632-02182-9); and Robert A. Meyers (ed.), Molecular Biology and Biotechnology, a Comprehensive Desk Reference, published by VCH Publishers, Inc., 1995 (ISBN 1-56081-569-8); Ausubel et al. (1987) Current Protocols in Molecular Biology, Green Publishing; Sambrook et al. (1989) Molecular Cloning: A Laboratory Manual, Cold Spring Harbor, N.Y.

In order to more fully understand the invention, the following definitions are provided:

As used herein, the term “allergen” refers to a protein or other compound from an organism such as an animal or plant capable of inducing an allergic response or an immune response in a patient. Known plant and animal allergens include, but are not limited to, milk, ragweed, wheat, barley, corn, rice, pigweed, soy, peanut, Brazil nut, English walnut, kiwis, citrus fruit, pollen extracts, dustmites, grass pollens, tree pollens (including oak and birch), mugwort, fish, shellfish such as shrimp, mussels and clams, cat dander, horse dander and eggs.

The term “genotype” refers to the genetic makeup of an organism such as a plant or animal. Plant and animal species generally have multiple genotypes. For example, barley which is used to produce barley flour for use in bread products, barley malt for use in beer, etc. has different genotypes such as ‘Harrington’, ‘Morex’, ‘Crystal’, ‘Stander’, ‘Moravian III’, ‘Gelena’, ‘Salome’, ‘Steptoe’, ‘Klages’ and ‘Baronesse’. Wheat, which is used to produce bread and other food products, has different genotypes such as ‘Anza’, ‘Karl’, ‘Bobwhite’ and ‘Yecora Rojo. Chickens (which produce eggs for direct consumption and use in food products in addition to being consumed directly) have different genotypes such as ‘Wyandotte’, ‘Rose Comb’, ‘Brahmas’ and ‘Pea Comb’. Cows, which produce milk in addition to being consumed directly, have different genotypes such as ‘Angus’, ‘Friesian’, ‘Continental’, ‘Charolais’ and ‘Blonde’. Peanuts used in such products as peanut butter and peanut oil in addition to being consumed directly have different genotypes such as PSB Pn 6, NSIC Pn 7, and NSIC Pn 8. Shrimp have different genotypes such as Penaeus monodon, P. vannamei, P. stylirostris, P. japanonicus.

The term “atopic dog colony” refers to an inbred colony of dogs which demonstrate an IgE-mediated response to common allergens, which can be readily assessed by means of titrated tests including, but not limited to: skin tests, feeding tests, gastroendoscopy tests, inhalation tests, and dermal patch tests.

The term “dermatitis” is intended to mean any of a large family of diseases of the skin that are characterized by inflammation of the skin attributable to a variety of etiologies (Dorland's Medical Dictionary). Dermatitis may be caused by inflammation to the skin including endogenous and contact dermatitis such as, but not limited to: actinic dermatitis (or photodermatitis), atopic dermatitis, chemical dermatitis, cosmetic dermatitis, dermatitis aestivalis, and seborrheic dermatitis.

As used herein, the term “transgenic animal” is intended to refer to an animal that has incorporated DNA sequences, including but not limited to genes which are perhaps not normally present, DNA sequences not normally transcribed into RNA or translated into a protein (“expressed”), or any other genes or DNA sequences which one desires to introduce into the non-transgenic animal, such as genes which may normally be present in the non-transgenic animal but which one desires to either genetically engineer or to have shared expression. The term also includes the offspring of the animals.

As used herein, the term “transgenic plant” is intended to refer to a plant that has incorporated DNA sequences, including but not limited to genes which are perhaps not normally present, DNA sequences not normally transcribed into RNA or translated into a protein (“expressed”), or any other genes or DNA sequences which one desires to introduce into the non-transformed plant, such as genes which may normally be present in the non-transformed plant but which one desires to either genetically engineer or to have shared expression. The term also includes the progeny of said plant or plant material, including seeds and plant cells. Thus, a plant that is grown from a plant cell into which recombinant DNA is introduced by transformation is a transgenic plant, as are all offspring of that plant that contain the introduced transgene, whether produced sexually or asexually.

As used herein, the term “crop plant” means any edible or non-edible plant grown for any commercial purpose, including, but not limited to the following purposes: cosmetics, seed production, hay production, ornamental use, fruit production, berry production, vegetable production, oil production, protein production, forage production, animal grazing, golf courses, lawns, flower production, landscaping, erosion control, green manure, improving soil health, producing pharmaceutical products/drugs, producing food additives, smoking products, pulp production and wood production. Thus, crop plants include floral plants, trees, and vegetable plants.

As used herein, the term “genetic construct” refers to the DNA or RNA molecule that comprises a nucleotide sequence which encodes the desired protein and which includes initiation and termination signals operably linked to regulatory elements including a promoter and polyadenylation signal capable of directing expression in the cells into which it is introduced.

The term “sensitization” is intended for the purpose of this invention to include the induction of acquired sensitivity or of allergy. Likewise, the term “sensitize” is intended for the purposes of this invention to render sensitive or to induce acquired sensitivity.

As used herein, “heterologous DNA” or “heterologous nucleic acid” includes DNA that does not occur naturally as part of the genome in which it is present or which is found in a location or locations in the genome that differs from that in which it occurs in nature. Heterologous DNA is not naturally occurring in that position or is not endogenous to the cell into which it is introduced, but has been obtained from another cell. Generally, although not necessarily, such DNA encodes proteins that are not normally produced by the cell in which it is expressed. Heterologous DNA can be from the same species or from a different species. Heterologous DNA may also be referred to as foreign DNA. Any DNA that one of skill in the art would recognize or consider as heterologous or foreign to the cell in which is expressed is herein encompassed by the term heterologous DNA. Examples of heterologous DNA include, but are not limited to, DNA that encodes test polypeptides, receptors, reporter genes, transcriptional and translational regulatory sequences, or selectable or traceable marker proteins, such as a protein that confers drug resistance.

The terms “heterologous protein”, “recombinant protein”, “exogenous protein”, and “protein of interest” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

The term “extract’ as used herein is intended to mean a concentrate of aqueous soluble plant components from the portion of the plant extracted and can be in aqueous or powdered form.

As used herein, the terms “allergic response” and “immune response” are used interchangeably and refer to an altered reactivity in response to an antigen and manifesting as various diseases, including, but not limited to, allergic rhinitis (seasonal or perennial, due to pollen or other allergens), asthma, polyps of the nasal cavity, unspecified nasal polyps, pharyngitis, nasopharyngitis, sinusitis, upper respiratory tract hypersensitivity reaction, gastrointestinal reactions and other allergies. Examples of allergies include, but are not limited to, anaphylaxis, allergic rhinitis (seasonal or perennial) or other respiratory allergy, food allergies and atopic skin reactions. Such responses can be Type I that are IgE-mediated immunologic reactions, or they can be Type II or type III that are IgA, IgG or IgM mediated reactions, or Type IV, cellular immune reactions.

The term “observe” is typically used to refer to a visual observation leading to a qualitative or quantitative determination or detection of an allergic response.

The term “organism” relates to any living entity comprised of at least one cell. An organism can be as simple as one prokaryotic cell or as complex as an animal.

The term “components of the pentose phosphate pathway” refers to components of pentose phosphate pathway involved in the oxidation of glucose-6-phosphate to yield NADPH and rebose-5 phosphate and in the conversion of ribose phosphates back to hexose phosphates allowing the oxidative reactions to continue. Such components include thioredoxin, NTR and glucose-6-phosphate dehydrogenase.

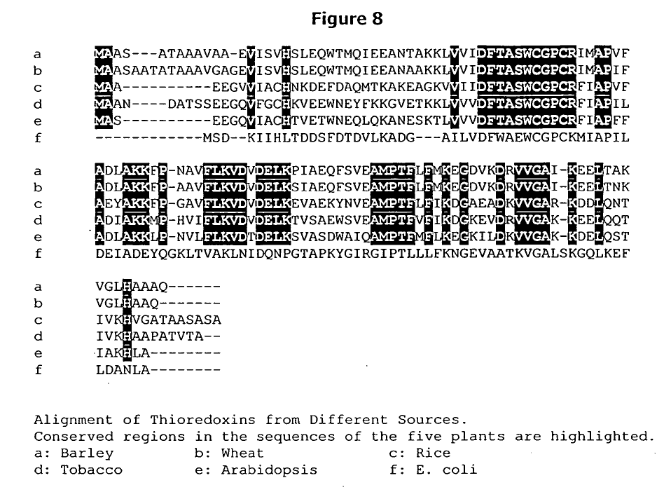

The term “thioredoxin protein or thioredoxin polypeptide” refers to a large number of plant, animal, and microbial thioredoxin proteins or polypeptides that have been characterized, and the genes encoding many of these proteins have been cloned and sequenced. The present invention is preferably directed to the use of thioredoxin h proteins, although other thioredoxin proteins may also be employed to produce transgenic plants as described herein. Among the thioredoxin h proteins from plants that have been described to date are thioredoxin h proteins from Spinacea oleracea (Florencio et al., 1988; Marcus et al., 1991); Arabidopsis thaliana (Rovera-Madrid et al., 1993; Rivera-Madrid et al., 1995), Nicotiana tabacum (Marty and Meyer, 1991; Brugidou et al., 1993), Oryza sativa (Ishiwatari et al., 1995), Brassica napus (Bower et al., 1996), Glycine max (Shi and Bhattacharyya, 1996), Triticum aestivum (Johnson et al., 1987; Gautier et al., 1998) and Hordeum vulgare (Calliau et al., 1998). The amino acid sequences of these and other thioredoxin h proteins, and the nucleotide sequence of cDNAs and/or genes that encode these proteins are available in the scientific literature and publicly accessible sequence databases. For example, a cDNA encoding thioredoxin h from Picea mariana is described in accession number AF051206 (NID g2982246) of GenBank, and located by a search using the Entrez browser nucleotide sequence search of the National Center for Biotechnology Information website located at ncbi.nim.nih.gov. The cDNA encoding the Triticum aestivum thioredoxin h protein is described on the same database under accession number X69915 (NID g2995377). In addition, particular thioredoxin sequences are shown in FIG. 8.

The present invention may be practiced using nucleic acid sequences that encode full length thioredoxin h proteins, as well as thioredoxin h derived proteins that retain thioredoxin h activity. Thioredoxin h derived proteins which retain thioredoxin biological activity include fragments of thioredoxin h, generated either by chemical (e.g., enzymatic) digestion or genetic engineering means; chemically functionalized protein molecules obtained starting with the exemplified protein or nucleic acid sequences, and protein sequence variants, for example allelic variants and mutational variants, such as those produced by in vitro mutagenesis techniques, such as gene shuffling (Stemmer et al., 1994a, 1994b). Thus, the term “thioredoxin h protein” encompasses full-length thioredoxin h proteins, as well as such thioredoxin h derived proteins that retain thioredoxin h activity.

Thioredoxin protein may be quantified in biological samples (such as seeds) either in terms of protein level, or in terms of thioredoxin activity. Thioredoxin protein level may be determined using a western blot analysis followed by quantitative scanning of the image as described elsewhere (Lozano et al., 1996). Thioredoxin activity may be quantified using a number of different methods known in the art. Preferred methods of measuring thioredoxin biological activity attributable to thioredoxin h in plant extracts include NADP/malate dehydrogenase activation (Johnson et al., 1987a,b) and reduction of 2′,5′-dithiobis (2-nitrobenzoic acid) (DTNB) via NADP-thioredoxin reductase (Florencio et al., 1988; U.S. Pat. No. 5,792,506). Due to the potential for interference from non-thioredoxin h enzymes that use NADPH, accurate determination of thioredoxin h activity should preferably be made using partially purified plant extracts. Standard protein purification methods, e.g., (NH₄)₂SO₄ extraction or heat can be used to accomplish this partial purification. The activity of thioredoxin h may also be expressed in terms of specific activity, i.e., thioredoxin activity per unit of protein present, as described in more detail below.

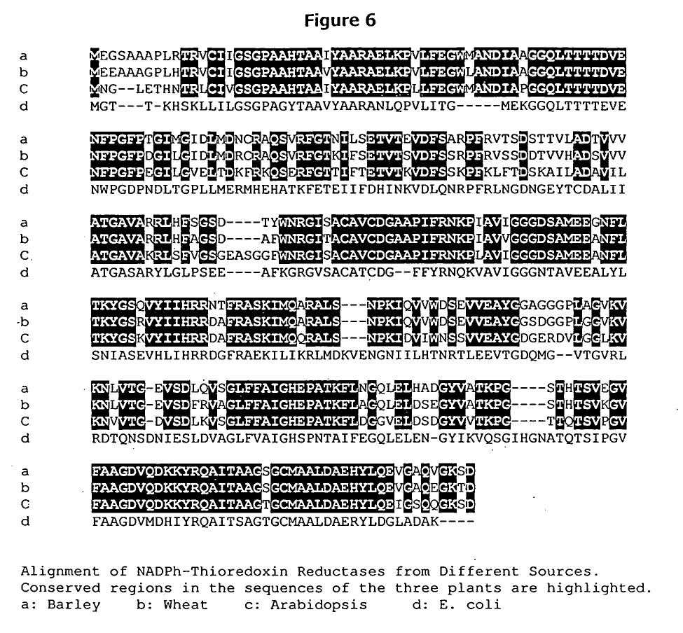

The term “NTR” refers to proteins capable of catalyzing the reduction of thioredoxin coupled to NADPH oxidation. NTR belongs to the pyridine nucleotide-disulfide oxidoreductase family which includes glutathione reductase, lipoamide reductase, etc., which catalyze the transfer of electrons from a pyridine nucleotide via a flavin carrier to, in most cases, disulfide-containing substrates. NTRs include those sequences described in FIG. 6 and homologues thereof.

The present invention may be practiced using nucleic acid sequences that encode full length NTR proteins, as well as NTR derived proteins that retain NTR activity. NTR derived proteins which retain NTR biological activity include fragments of NTR, generated either by chemical (e.g. enzymatic) digestion or genetic engineering means; chemically functionalized protein molecules obtained starting with the exemplified protein or nucleic acid sequences, and protein sequence variants, for example allelic variants and mutational variants, such as those produced by in vitro mutagenesis techniques, such as gene shuffling (Stemmer et al, 1994a, 1994b). Thus, the term “NTR protein” encompasses full-length NTR proteins, as well as such NTR derived proteins that retain NTR activity.

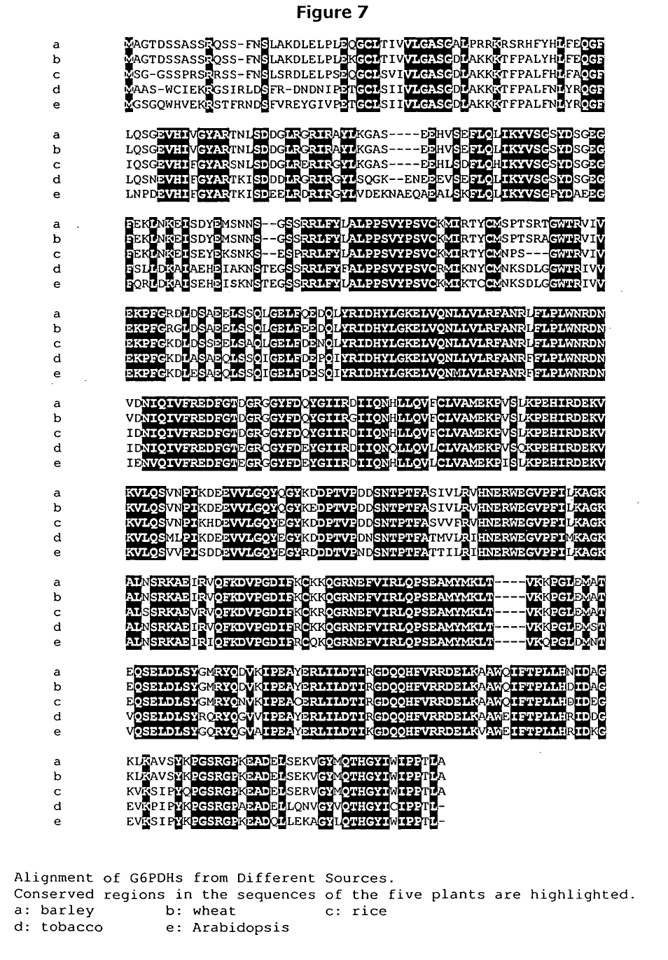

The term glucose-6-phosphate dehydrogenase, (G6PDH) refers to an enzyme that catalyzes the first step of the oxidative pentose phosphate pathway (OPPP), namely the conversion of glucose-6-phosphate to 6-phosphogluconolactone. Concomitantly, NADPH is generated. The main function of G6PDH is to generate NADPH for anabolic metabolism, including fatty acid, amino acid and ribose synthesis. G6PDH includes those sequences described in FIG. 7 and homologues thereof.

The present invention may be practiced using nucleic acid sequences that encode full length G6PDH proteins, as well as G6PDH derived proteins that retain G6PDH activity. G6PDH derived proteins which retain G6PDH biological activity include fragments of G6PDH, generated either by chemical (e.g. enzymatic) digestion or genetic engineering means; chemically functionalized protein molecules obtained starting with the exemplified protein or nucleic acid sequences, and protein sequence variants, for example allelic variants and mutational variants, such as those produced by in vitro mutagenesis techniques, such as gene shuffling (Stemmer et al., 1994a, 1994b). Thus, the term “G6PDH protein” encompasses full-length G6PDH proteins, as well as such G6PDH derived proteins that retain G6PDH activity.

A “promoter” refers to a regulatory nucleic acid sequence, typically located upstream (5′) of a gene that, in conjunction with various cellular proteins, is responsible for regulating the expression of the gene. Promoters may regulate gene expression in a number of ways. For example, the expression may be tissue-specific, meaning that the gene is expressed at enhanced levels in certain tissues, or developmentally regulated, such that the gene is expressed at enhanced levels at certain times during development, or both.

The expression of a transgene in seeds or grains according to the present invention is preferably accomplished by operably linking a seed-specific or grain-specific promoter to the nucleic acid molecule encoding the transgene protein. In this context, “seed-specific” indicates that the promoter has enhanced activity in seeds compared to other plant tissues; it does not require that the promoter is solely active in the seeds. Accordingly, “grain-specific” indicates that the promoter has enhanced activity in grains compared to other plant tissues; it does not require that the promoter is solely active in the grain. Preferably, the seed- or grain-specific promoter selected will, at the time when the promoter is most active in seeds, produce expression of a protein in the seed of a plant that is at least about two-fold greater than expression of the protein produced by that same promoter in the leaves or roots of the plant. However, given the nature of the thioredoxin protein, it may be advantageous to select a seed- or grain-specific promoter that causes little or no protein expression in tissues other than seed or grain. In a preferred embodiment, a promoter is specific for seed and grain expression, such that, expression in the seed and grain is enhanced as compared to other plant tissues but does not require that the promoter be solely active in the grain and seed. In a preferred embodiment, the promoter is “specific” for a structure or element of a seed or grain, such as an embryo-specific promoter. In accordance with the definitions provided above, an embryo-specific promoter has enhanced activity in an embryo as compared to other parts of a seed or grain or a plant and does not require its activity to be limited to an embryo. In a preferred embodiment, the promoter is “maturation-specific” and accordingly has enhanced activity developmentally during the maturation of a part of a plant as compared to other parts of a plant and does not require its activity to be limited to the development of a part of a plant.

A seed- or grain-specific promoter may produce expression in various tissues of the seed, including the endosperm, embryo, and aleurone or grain. Any seed- or grain-specific promoter may be used for this purpose, although it will be advantageous to select a seed- or grain-specific promoter that produces high level expression of the protein in the plant seed or grain. Known seed- or grain-specific promoters include those associated with genes that encode plant seed storage proteins such as genes encoding: barley hordeins, rice glutelins, oryzins, or prolamines; wheat gliadins or glutenins; maize zeins or glutelins; maize embryo-specific promoter; oat glutelins; sorghum kafirins; millet pennisetins; or rye secalins.

In certain embodiments, the seed- or grain-specific promoter that is selected is a maturation-specific promoter. The use of promoters that confer enhanced expression during seed or grain maturation (such as the barley hordein promoters) may result in even higher levels of thioredoxin expression in the seed.

By “seed or grain-maturation” herein refers to the period starting with fertilization in which metabolizable food reserves (e.g., proteins, lipids, starch, etc.) are deposited in the developing seed, particularly in storage organs of the seed, including the endosperm, testa, aleurone layer, embryo, and scutellar epithelium, resulting in enlargement and filling of the seed and ending with seed desiccation.

Members of the grass family, which include the cereal grains, produce dry, one-seeded fruits. This type of fruit is, strictly speaking, a caryopsis but is commonly called a kernel or grain. The caryopsis of a fruit coat or pericarp surrounds the seed and adheres tightly to a seed coat. The seed consists of an embryo or germ and an endosperm enclosed by a nucellar epidermis and a seed coat. Accordingly the grain comprises the seed and its coat or pericarp. The seed comprises the embryo and the endosperm. (R. Carl Hoseney in “Principles of Cereal Science and Technology” expressly incorporated by reference in its entirety.)

“Sequence identity” refers to the similarity between two nucleic acid sequences, or two amino acid sequences is expressed in terms of sequence identity (or, for proteins, also in terms of sequence similarity). Sequence identity is frequently measured in terms of percentage identity; the higher the percentage, the more similar the two sequences are. As described above, homologs and variants of the thioredoxin nucleic acid molecules, hordein promoters and hordein signal peptides may be used in the present invention. Homologs and variants of these nucleic acid molecules will possess a relatively high degree of sequence identity when aligned using standard methods.

Methods of alignment of sequences for comparison are well known in the art. Various programs and alignment algorithms are described in: Smith and Waterman (1981); Needleman and Wunsch (1970); Pearson and Lipman (1988); Higgins and Sharp (1988); Higgins and Sharp (1989); Corpet et al., (1988); Huang et al., (1992); and Pearson et al, (1994). Altschul et al., (1994) presents a detailed consideration of sequence alignment methods and homology calculations.

The NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the web site located at ncbi.nlm.nlh.gov/BLAST. A description of how to determine sequence identity using this program is available at the web site located at nchi.nlm.nih.gov/BLAST/blast.help

Homologs of the disclosed protein sequences are typically characterized by possession of at least 40% sequence identity counted over the full length alignment with the amino acid sequence of the disclosed sequence using the NCBI Blast 2.0, gapped blastp set to default parameters. The adjustable parameters are preferably set with the following values: overlap span 1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. Proteins with even greater similarity to the reference sequences will show increasing percentage identities when assessed by this method, such as at least about 50%, at least about 60%, at least about 70%, at least about 75%, at least about 80%, at least about 90% or at least about 95% sequence identity.

Homologs of the disclosed nucleic acid sequences are typically characterized by possession of at least 40% sequence identity counted over the full length alignment with the amino acid sequence of the disclosed sequence using the NCBI Blast 2.0, gapped blastn set to default parameters. A preferred method utilizes the BLASTN module of WU-BLAST-2 (Altschul et al. 1996); set to the default parameters, with overlap span and overlap fraction set to 1 and 0.125, respectively. Nucleic acid sequences with even greater similarity to the reference sequences will show increasing percentage identities when assessed by this method, such as at least about 50%, at least about 60%, at least about 70%, at least about 75%, at least about 80%, at least about 90% or at least about 95% sequence identity.

The alignment may include the introduction of gaps in the sequences to be aligned. In addition, for sequences which contain either more or fewer amino acids than the protein encoded by the sequences in the figures, it is understood that in one embodiment, the percentage of sequence identity will be determined based on the number of identical amino acids in relation to the total number of amino acids. Thus, for example, sequence identity of sequences shorter than that shown in the figures as discussed below, will be determined using the number of amino acids in the longer sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described herein for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As will be appreciated by those skilled in the art, the sequences of the present invention may contain sequencing errors. That is, there may be incorrect nucleosides, frameshifts, unknown nucleosides, or other types of sequencing errors in any of the sequences; however, the correct sequences will fall within the homology and stringency definitions herein.

A “vector” refers to a nucleic acid molecule as introduced into a host cell, thereby producing a transformed host cell. A vector may include one or more nucleic add sequences that permit it to replicate in one or more host cells, such as origin(s) of replication. A vector may also include one or more selectable marker genes and other genetic elements known in the art.

A “transformed” cell is a cell into which has been introduced a nucleic acid molecule by molecular biology techniques. As used herein, the term transformation encompasses all techniques by which a nucleic acid molecule might be introduced into such a cell, plant or animal cell, including transfection with viral vectors, transformation by Agrobacterium, with plasmid vectors, and introduction of naked DNA by electroporation, lipofection, and particle gun acceleration and includes transient as well as stable transformants.

An “isolated” biological component (such as a nucleic acid or protein or organelle) has been substantially separated or purified away from other biological components in the cell or the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term embraces nucleic acids including chemically synthesized nucleic acids and also embraces proteins prepared by recombinant expression in vitro or in a host cell and recombinant nucleic acids as defined below.

“Operably linked” refers to a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary, join two protein-coding regions in the same reading frame. With respect to polypeptides, two polypeptide sequences may be operably linked by covalent linkage, such as through peptide bonds or disulfide bonds.

By “recombinant nucleic acid” herein is meant a nucleic acid that has a sequence that is not naturally occurring or has a sequence that is made by an artificial combination of two otherwise separated segments of sequence. This artificial combination is often accomplished by chemical synthesis or, more commonly, by the artificial manipulation of nucleic acids, e.g., by genetic engineering techniques, such as by the manipulation of at least one nucleic acid by a restriction enzyme, ligase, recombinase, and/or a polymerase. Once introduced into a host cell, a recombinant nucleic acid is replicated by the host cell, however, the recombinant nucleic acid once replicated in the cell remains a recombinant nucleic acid for purposes of this invention. By “recombinant protein” herein is meant a protein produced by a method employing a recombinant nucleic acid. As outlined above “recombinant nucleic acids” and “recombinant proteins” also are “isolated” as described above.

A “complementary DNA, (cDNA)” is a piece of DNA that is synthesized in the laboratory by reversed transcription of an RNA, preferably an RNA extracted from cells. cDNA produced from mRNA typically lacks internal, non-coding segments (introns) and regulatory sequences that determine transcription.

An “open reading frame, (ORF)” is a series of nucleotide triplets (codons) coding for amino acids without any internal termination codons. These sequences are usually translatable into a peptide.

A “reduced protein” is a protein in which the disulfide (S—S) group(s) resulting from oxidized cysteine (cystine) residues is converted to the sulfhydryl (2 SH) state by the enzymatic transfer of reducing equivalents from a cofactor (NADPH) or a protein (reduced ferredoxin) in the presence of an enzyme. Such a protein can also be reduced nonenzymatically by a chemical agent such as dithiothreitol.

A “transgenic plant” refers to a plant that contains recombinant genetic material not normally found in plants of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant and parts of said plant, for instance grains, seeds, flowers, leaves, roots, fruit, pollen, stems, etc.

The term purified does not require absolute purity: rather, it is intended as a relative term. Thus, for example, a purified barley thioredoxin h protein preparation is one in which the barley thioredoxin h protein is more enriched or more biochemically active or more easily detected than the protein is in its natural environment within a cell or plant tissue. Accordingly, “purified” embraces or includes the removal or inactivation of an inhibitor of a molecule of interest. In a preferred embodiment, a preparation of barley thioredoxin h protein is purified such that the barley thioredoxin h represents at least 5-10% of the total protein content of the preparation. For particular applications, higher protein purity may be desired, such that preparations in which barley thioredoxin h represents at least 50% or at least 75% or at least 90% of the total protein content may be employed.

Two nucleotide or amino acid sequences are orthologs of each other if they share a common ancestral sequence and diverged when a species carrying that ancestral sequence split into two species, sub-species, or cultivars. Orthologous sequences are also homologous sequences. The term “polynucleotide,” “oligonucleotide,” or “nucleic acid” refers to a polymeric form of nucleotides of any length, either deoxyribonucleotides or ribonucleotides, or analogs thereof. The terms “polynucleotide” and “nucleotide” as used herein are used interchangeably. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A “fragment” or “segment” of a nucleic acid is a small piece of that nucleic acid.

A “gene” refers to a polynucleotide containing at least one open reading frame that is capable of encoding a particular protein after being transcribed and translated.

The terms “primer” and “nucleic acid primer” are used interchangeably herein. A “primer” refers to a short polynucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a polymerase and at a suitable temperature and pH. The primer may be either single-stranded or double-stranded and must be sufficiently long to prime the synthesis of the desired extension product. The exact length of the primer will depend upon many factors, including temperature, source of primer and use of the method.

A “polymerase chain reaction” (“PCR”) is a reaction in which replicate copies are made of a target polynucleotide using a “primer pair” or a “set of primers” consisting of an “forward” and a “reverse” primer, and a catalyst of polymerization, such as a DNA polymerase, and particularly a thermally stable polymerase enzyme. Methods for PCR are taught in U.S. Pat. No. 4,683,195 (Mullis) and U.S. Pat. No. 4,683,202 (Mullis et al.). All processes of producing replicate copies of the same polynucleotide, such as PCR or gene cloning, are collectively referred to herein as “amplification” or “replication”.

Taking into account these definitions, the present invention relates to the selection of low allergen plant and animal genotypes for production of low allergen food products. Once selected, these low allergen plant and animal genotypes can be made even less allergic through transgenic or conventional breeding technologies.

Allergic Reactions to Food Products

Food normally doesn't provoke a response from the human immune system, the body's defense against microbes and other threats to health. In food allergies, two parts of the immune response are generally involved (i) the production of immunoglobulin E (IgE) antibodies that circulate in the blood and interact with (ii) mast cells. Mast cells occur in all body tissues but especially in areas that are typical sites of allergic reactions, including the nose, throat, lungs, skin, and gastrointestinal tract.

People usually inherit the ability to form IgE against food. Those more likely to develop food allergies come from families in which allergies such as hay fever, asthma, or eczema are common.

A predisposed person must first be exposed to a specific food before IgE is formed. As this food is digested for the first time, tiny protein fragments prompt certain cells to produce specific IgE against that food. The IgE then attaches to the surface of mast cells. The next time the particular food is eaten, the protein interacts with the specific IgE on the mast cells and triggers the release of chemicals such as histamine that produce the symptoms of an allergic reaction.

If the mast cells release chemicals in the nose and throat, the allergic person may experience an itching tongue or mouth and may have trouble breathing or swallowing. If mast cells in the gastrointestinal tract are involved, the person may have diarrhea or abdominal pain. Skin mast cells can produce hives or intense itching.

The food protein fragments responsible for an allergic reaction are not broken down by cooking or by stomach acids or enzymes that digest food. These proteins can cross the gastrointestinal lining, travel through the bloodstream and cause allergic reactions throughout the body.

The timing and location of an allergic reaction to food is affected by digestion. For example, an allergic person may first experience a severe itching of the tongue or “tingling lips.” Vomiting, cramps or diarrhea may follow. Later, as allergens enter the bloodstream and travel throughout the body, they can cause a drop in blood pressure, hives or eczema, or asthma when they reach the lungs. The onset of these symptoms may vary from a few minutes to an hour or two after the food is eaten.

Food allergy patterns in adults differ somewhat from those in children. The most common foods to cause allergies in adults are shrimp, lobster, crab, and other shellfish; peanuts (one of the chief foods responsible for severe anaphylaxis); walnuts and other tree nuts; fish; and eggs.

In children, eggs, milk, peanuts, soy and wheat are the main culprits. Children typically outgrow their allergies to milk, egg, soy and wheat, while allergies to peanuts, tree nuts, fish and shrimp usually are not outgrown. Adults usually do not lose their allergies.

Prior to the present invention, it was assumed that if an individual was allergic to a specific food, plant or animal such as milk, egg, soy and wheat, peanuts, tree nuts, fish, shrimp, etc. they were similarly allergic to all plant and animal genotypes that produced the food. For example, prior to this invention, it was not thought that different wheat genotypes would produce wheat food products that caused different levels of an allergenic response, that different cow genotypes would produce milk with different levels of an allergenic response, different chicken genotypes would produce eggs with different levels of an allergenic response, etc.

As such, the invention includes, in one aspect, a method of determining the allergenicity of a plant or animal genotype compared to a mixture or collection of different plant or animal genotypes. It has been discovered that different genotypes produce different allergenic responses.

In another aspect, the invention includes a method for determining the allergenicity of a plant or animal subgroup or subspecies compared to a mixture of different plant or animal subgroups or subspecies. One example of a subgroup would be two or more genotypes that produce similar degrees of allergic responses.

One of skill in the art will understand that there are many ways of comparing the allergenicity of different genotypes, subgroups or subspecies. This invention is not limited by the particular method of comparison of allergic reaction. Furthermore, one of skill in the will recognize that the methods of the present invention are applicable to human allergies as well as other animal allergies. For example, some dogs and cats are known to suffer from food allergies. Some dog breeds appear to have a genetic predisposition for allergies, including Retrievers, Cocker Spaniels, Sharpeis, Dalmatians, Poodles, Shepherds, Boxers and Bulldogs.

Preparation of Plant and Animal Genotypes for Allergy Testing

For some plant and animal genotypes, a sample of the plant, animal, or product thereof may be used directly in allergen tests. For others, the allergenic protein or compound will need to be extracted prior to testing. This will depend in part upon the severity of the allergic reaction to the allergen and in part upon the sensitivity of the allergen test used.

Protein-containing extracts are prepared from various plant and animal genotypes for allergy testing by general procedures well known in the art as described in Protein Purification: Principles, High-Resolution Methods, and Applications, 2nd Edition Jan-Christer Janson, Lars Ryden, March 1998. Ideally the genotypes are grown under the same or similar conditions because the growth conditions could alter the ratios of allergenic and non-allergenic proteins. For example, in plants such as wheat, barley, rice, peanuts, etc., the protein-containing extracts are prepared by grinding, mashing or otherwise breaking the plant up into pieces prior to protein extraction into buffer. For chickens, the eggs from different genotypes of chickens are harvested and the egg proteins are extracted. For cows, the milk from different genotypes of cows is collected and concentrated to further purify the proteins.

Once the protein extracts are isolated, they can be tested in various allergy test such as skin testing including prick and injection methods; oral challenge tests; blood testing including RAST assays, IgE immunoblot enzyme linked immunosorbent assays (ELISA), radio-immunoassays (RIA), “sandwich” immunoradiometric assays (IRMA), enzyme-labeled immunodot assays and dog testing.

Skin Testing

There are two general approaches to allergy skin testing—the prick and the injection methods. In the prick method, a drop of extract is introduced using a small sharp instrument, causing a small break in the skin. With the injection method, a drop of allergen extract is injected into the top layer of the skin, raising a small bubble on its surface. Both of these tests are simple and inexpensive. The prick method has advantages in that it's safe, causes very little discomfort to the patient and allows medical personnel to test many allergens in one session.

In either method, the allergy extract causes a reaction in the skin in about 20 minutes. A negative reaction shows no change, while a positive reaction causes a small red welt to develop. The size of the welt is measured to determine the strength of the reaction.

The skin test may be performed upon humans or animals that are allergic to the particular allergen. In some cases, an alternative animal model may be used. For example, atopic dogs may be used as an animal model for human allergies.

For the skin prick test, a tiny amount of allergen is lightly pricked into the superficial skin. If a patient has an allergy, the specific allergen that the patient is allergic to will cause a chain reaction to begin in the patient's body. The spot where the allergen entered the skin will swell and itch a bit, forming a hive smaller than a quarter. The test results are generally available within 15 minutes of testing and the small hives where the test was done go away within 30 minutes.

The intradermal test involves injecting a tiny amount of allergen under the skin, usually on the upper arms or the abdomen of dogs.

Oral Challenge Tests

Challenge tests involve having a patient inhale or swallow a very small amount of the suspected allergen, such as milk or an antibiotic. If there is no reaction, the dose may be slowly increased. Since challenge tests may induce severe allergic reactions, they are only done when absolutely necessary, and must be closely supervised by an allergist.

Blood Tests

A patient's blood may be analyzed to determine sensitivity to various antigens using various immunoassay techniques. These methods include, but are not limited to, radioallergosorbent (RAST) inhibition tests, IgE immunoblot enzyme linked immunosorbent assays (ELISA), radio-immunoassays (RIA), “sandwich” immunoradiometric assays (IRMA), and enzyme-labeled immunodot assays as described in Antibody Techniques, V. Malik and E. Ullehoj Editors, 1994 Academic Press.

A wide range of immunoassay techniques are available as can be seen by reference to U.S. Pat. Nos. 4,015,043, 4,424,279 and 4,018,653 each of which is hereby incorporated by reference. This includes both single-site and two-site, or “sandwich”, assays of the non-competitive types, as well as in the traditional competitive binding assays. Sandwich assays are among the most useful and commonly used assays. A number of variations of the sandwich assay technique exist, and all are intended to be encompassed by the present invention. Briefly, in a typical forward assay, an unlabeled antibody is immobilized in a solid substrate and the sample to be tested brought into contact with the bound molecule. After a suitable period of incubation, for a period of time sufficient to allow formation of an antibody-antigen secondary complex, a second antibody, labeled with a reporter molecule capable of producing a detectable signal is then added and incubated, allowing time sufficient for the formation of a tertiary complex of antibody-antigen-labeled. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labeled antibody are added simultaneously to the bound antibody, or a reverse assay in which the labeled antibody and sample to be tested are first combined, incubated and then added simultaneously to the bound antibody. These techniques are well known to those skilled in the art, including any minor variations as will be readily apparent.

In the typical forward sandwich assay, a first antibody having specificity for a specific allergen, or antigenic parts thereof, contemplated in this invention, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated at 25° C. for a period of time sufficient to allow binding of any subunit present in the antibody. The incubation period will vary but will generally be in the range of about 2-40 minutes. Following the incubation period, the antibody subunit solid phase is washed and dried and incubated with a second antibody specific for a portion of the hapten. The second antibody is linked to a reporter molecule which is used to indicate the binding of the second antibody to the hapten.

By “reporter molecule,” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e., radioisotopes). In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. For example, R-nitrophenyl phosphate is suitable for use with alkaline phosphatase conjugates; for peroxidase conjugates, 1,2-phenylenediamine, 5-aminosalicylic acid, or toluidine are commonly used. It is also possible to employ fluorogenic substrates, which yield a fluorescent product rather than the chromogenic substrates noted above. In all cases, the enzyme-labeled antibody is added to the first antibody hapten complex, allowed to bind, and then the excess reagent is washed away. A solution containing the appropriate substrate is then added to the tertiary complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells or latex beads, and the like.

Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state of excitability in the molecule, followed by emission of the light at a characteristic color visually detectable with a light microscope. As in the EIA, the fluorescent labeled antibody is allowed to bind to the first antibody-hapten complex. After washing off the unbound reagent, the remaining tertiary complex is then exposed to the light of the appropriate wavelength, the fluorescein observed indicates the presence of the hapten of interest. Immunofluorescence and EIA techniques are both very well established in the art and are particularly preferred for the present method. However, other reporter molecules, such as radioisotope, chemilluminescent or bioluminescent molecules, may also be employed. It will be readily apparent to the skilled technician how to vary the procedure to suit the required purpose.

Kits for these immunoassays are commercially available from vendors including CMG™ (Fribourg, Switzerland) and Antibodies Inc.™ (Davis, Calif.). Blood samples are taken from a patient and immunoassays are performed to determine if the patient has an immune response to a particular antigen.

Dog Tests

Dog colony tests employ a newborn dog of an atopic colony having a number of special characteristics. The dogs in the atopic colony are inbred, and are selected for a genetic predisposition to an allergy. The dogs may have a history of sensitivity to pollens or foods, and can be of a variety of breeds. Preferably, the dogs are spaniels or basenji dogs or mixed breed spaniel/Basenji dogs. However, the dogs are not limited to these breeds. Once the dogs are produced, they can be bred, inbred, crossbred or outbred to produce further atopic colonies for use as dog models according to the present invention.

The dogs have a history of sensitivity to pollens or foods. The sensitivity can be detected using standard immunometric methods to detect serum IgE levels in the dog.

These methods include, but are not limited to, IgE immunoblot enzyme linked immunosorbent assays (ELISA), radio-immunoassays (RIA), “sandwich” immunoradiometric assays (IRMA), and enzyme-labeled immunodot assays. Kits for these assays are commercially available from vendors including CMG™ (Fribourg, Switzerland) and Antibodies Inc.™ (Davis, Calif.).

Methods for performing an immunodot assay for identifying atopic dogs in accordance with the invention can be found in Ermel et al., 1997. Typically, the immunodot assay involves aliquoting food antigen extracts onto nitrocellulose strips that are then blocked with casein or ovalbumin to prevent nonspecific protein adsorption. The strips are then incubated at 4° for 18 hours in serum from the dog which has been diluted, followed by a 1 hour incubation with a primary anti-canine IgE antibody at room temperature. Bound antibodies can then be detected by incubating with anti-primary antibody immunoglobulins that are coupled to a detectable marker. Examples of suitable detectable markers include but are not limited to: enzymes, coenzymes, enzyme inhibitors, chromophores, fluorophores, chemiluminescent materials, paramagnetic metals, spin labels, and radionuclides. The strips can then be developed and quantitated by standard methods.

As seen in the present invention, dogs sensitized to an allergen from a single source (for example, wheat) can be used for testing allergens from a related source (barley or other cereals). This feature greatly broadens the use of the dog colony for testing foods or other allergenic materials.

Sensitizing, Challenging, and Observing Stage

The first step of the dog testing method involves sensitizing a newborn dog from an atopic colony with an extract by injecting into, feeding, or applying to the skin, the extract to the newborn dog.

The second step of the method involves challenging the dog with the extract after a period sufficient to allow the dog to establish an immune response, and observing the degree of allergic response provoked or no response. The various methods used for challenging and observing allergic responses in the dog include skin tests, feeding tests, gastroendoscopy tests, inhalation tests and transdermal patch tests.

Skin Test

The skin test may be used to challenge the dog by applying the allergen material to a skin region of the dog and observing local wheal formation at the application site as the allergic response. Procedures for skin tests to measure the allergic hypersensitivity reaction are described in Ermel et al., 1997, Buchanan et al., 1997, and del Val et al., 1999.

Feeding Test

The feeding test may be used to challenge the dog by feeding the allergen material to the dog, and observing gastrointestinal upset as the allergic response. Sensitized pups challenged orally with food allergens may respond with clinical manifestations of food allergy including loose “mud-pie” diarrhea, occasional nausea and vomiting. Signs of nausea and vomiting may be acute, often observed immediately or within 12 hours of food antigen exposure and may be resolved in up to about 4 days.

Gastroendoscopy Test

The gastroendoscopy test is used to challenge the dog by contacting the allergen material directly with the wall or injecting into the stomach of the dog and observing as the allergic response a local wheal at 3 minutes after contact and inflammation at 24 hours after contact at the application site. Procedures for gastroendoscopy tests are described in Ermel et al., 1997. Generally, on the day before endoscopy the dogs are fed a hypoallergenic liquid maintenance elemental diet. The dogs are premedicated with atropine to minimize gastrointestinal tract secretions during the procedure. Anesthesia can be induced with Telazol (Aveco Co., Inc., Fort Dodge, Iowa) to allow intubation. Dogs are positioned in sternal recumbency for the endoscopic examinations.

The endoscopy procedure can be performed with a Pentax upper gastrointestinal tract endoscope (Pentax, Orangeburg, N.Y.) which can be fitted with an ultra miniature endoscopic video camera. Food antigen extracts are injected into the gastric mucosa via needles passed through the biopsy channel of the endoscope.

Food allergen extracts are administered into the gastric mucosa along the ventral-lateral aspect of the greater curvature of the stomach near the confluence with the pyloric antrum. A series of dilutions of known antigens can be injected into the gastric mucosa to determine the optimal concentration for gastroscopic food sensitivity testing. A mixture of physiologic saline and glycerin can be used as a control. Approximately 5 to 10 minutes before the injections filtered 0.5% (w/v) Evans blue dye solution can be given intravenously to enhance visualization of the allergic response (0.2 ml/kg animal weight).

Gastric mucosal tissue specimens are collected before food extract and control injections with radial jaw biopsy forceps. Gastric mucosal responses are graded according to the amount of swelling, erythema, and blue patching that is observed about 3 minutes after the injection of food extract or control. The injection sites are continuously observed and videotaped for 3 minutes after each injection and biopsy specimens can be obtained immediately after the 3 minute observation period. The injection sites can be re-examined and videotaped at 15 to 30 minutes and 24 to 48 hours after the injections. Additional gastric mucosal tissue specimens are collected from the dogs 24 to 48 hours after injection. The biopsy tissue specimens can be fixed in buffered 10% formalin for histologic examination. The videotapes are reviewed and graded by persons unaware of the identity and order of the injected food antigen extracts.

Inhalation Test and Transdermal Patch Test

The inhalation test may be used to challenge the dog by administering the allergen material by inhalation to the dog, and observing bronchial constriction as the allergic response. A transdermal patch may be used by applying the allergen material with a patch immobilized on the skin and observing inflammation after 24 to 72 hr at the site of application. Both of these methods are standard to one skilled in the art.

The third step of the method involves determining whether a detectable skin reaction has been observed after following the first and second steps described above.

Qualitative Analysis

In one embodiment, if a detectable skin reaction is observed, then the sensitizing, challenging and observing steps carried out above are repeated using a second plant or animal extract from a second genotype. The degree of the two skin reactions are then compared to one another.

The degree of allergic response produced by the test material may be graded by sensitizing the dog with at least two different allergens known to provoke a different degree of allergic response in humans and one non-allergen, challenging the dog with each of at least two different known allergens, thus to determine the degree of immune response associated with the different known allergens, and if an allergic response is observed following the challenge with the two different allergens and with the test substance, but not with the control material, then matching the degree of response to the test allergen with one or more of the responses observed in the challenging step with the known allergens.

Screening for Low Allergen Growth Conditions

The conditions under which a plant or animal are grown can influence the ratio of the allergenic and non-allergenic proteins. The methods of the present invention may be used to screen for particular conditions that produce low allergic reactions

Plant Growth

One of skill in the art will recognize that any condition could be tested for its effect on allergenicity of the plant produced. Examples of such conditions include but are not limited to temperature, lighting, time of planting, time of harvesting, composition of fertilizer, watering regimen, and soil conditions. Once the plants have been grown, the allergenidty may be tested as described above.

Animal Growth

One of skill in the art will recognize that any condition could be tested for its effect on allergenicity of the animal produced. Examples of such conditions include but are not limited to temperature, feeding regimen, including amount, types of food, and timing of feeding, and degree of exercise permitted. Again, once the animals have been grown, the allergenicity may be tested as described above.

Use of the Selected Low Allergen Genotypes

Once a reduced allergen plant or animal genotype has been selected, it can be consumed directly or utilized in a variety of ways to produce a low allergen food product. Such food products are produced by procedures well known in the art. Alternatively, the selected low or reduced allergen genotype can be made even less allergenic through traditional breeding techniques or transgenic technologies.

Plant Breeding

Low allergen inducing selected plants may be made even less allergenic by use of traditional plant breeding techniques. Such techniques are well known in the art and include those described in Plant Breeding: Theory and Techniques S. K. Gupta, Editor. Jodhpur, Agrobios, 2000, 388.

Transgenic Plants

Methods for producing transgenic plants for both monocots and dicots are currently available and known to those of skill in the art.

A variety of expression vectors can be used to transfer a gene encoding plant pentose phosphate pathway proteins including thioredoxin, NTR or G6PDH as well as the desired promoters and regulatory proteins into a plant. Examples include but not limited to those derived from a Ti plasmid of Agrobacterium tumefaciens, as well as those disclosed by Herrera-Estrella, L., et al., Nature 303: 209 (1983), Bevan, M., Nucl. Acids Res. 12: 8711-8721 (1984), Klee, H. J., Bio/Technology 3: 637-642 (1985), and EPO Publication 120,516 (Schilperoort et al.) for plantyledonous plants and monocotyleclonous plants (M. Uze, et al. Plant Science 130:87 (1997). Alternatively, non-Ti vectors can be used to transfer the DNA constructs of this invention into monocotyledonous plants and plant cells by using free DNA delivery techniques. Such methods may involve, for example, the use of liposomes, electroporation, microprojectile bombardment, silicon carbide whiskers, viruses and pollen. By using these methods transgenic plants such as wheat, rice (Christou, P., Bio/Technology 9: 957-962 (1991)) and corn (Gordon-Kamm, W., Plant Cell 2: 603-618 (1990)) are produced.

After transformation of cells or protoplasts, the choice of methods for regenerating fertile plants is not particularly important. Suitable protocols are available for Leguminosae (alfalfa, soybean, clover, etc.), Umbelliferae (Carrot, celery, parsnip), Crudferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture Crop Species. Macmillan Publ. Co.; Shimamoto et al. (1989) Nature 338:274-276; Fromm et al. (1990) Bio/Technology 8:833-839; Vasil et al. (1990) Bio/Technology 8:429434 and peanuts, Li, Z. et al., Development of Gene Delivery Systems Capable of Introducing Aspergillus flavus—Resistance Genes into Peanuts, p. 12, Proceedings from the 7th Annual Aflatoxin Elim Workshop Meeting, St. Louis, Mo., J. Robens (ed.), USDA-ARS, Beltsville, Md. 20705, 1994; Ozias-Akins, P. et al., Genetic Engineering of Peanut-Insertion of Four Genes that May Offer Disease Resistance Strategies p. 14, Proceedings from the 7th Annual Aflatoxin Elim Workshop Meeting, St. Louis, Mo., Robens (ed.), USDA-ARS, Beltsville, Md. 20705, 1994; Weissinger, A. et al., Progress in the Development of Transgenic Peanut with Enhanced Resistance to Fungi, p. 13, Proceedings from the 7th Annual Aflatoxin Elim Workshop Meeting, St. Louis, Mo., Robens (ed.), USDA-ARS, Beltsville, Md. 20705, 1994.

Animals

In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome.

Animals for obtaining eggs or other nucleated cells (e.g. embryonic stem cells) for generating transgenic animals can be obtained from standard commercial sources such as Charles River Laboratories (Wilmington, Mass.), Taconic (Germantown, N.Y.), Harlan Sprague Dawley (Indianapolis, Ind.).

Eggs can be obtained from suitable animals, e.g., by flushing from the oviduct or using techniques described in U.S. Pat. No. 5,489,742 issued Feb. 6, 1996 to Hammer and Taurog; U.S. Pat. No. 5,625,125 issued on Apr. 29, 1997 to Bennett et al.; Gordon et al., 1980, Proc. Natl. Acad. Sci. USA 77:7380-7384; Gordon & Ruddle, 1981, Science 214: 1244-1246; U.S. Pat. No. 4,873,191 to T. E. Wagner and P. C. Hoppe; U.S. Pat. No. 5,604,131; Armstrong, et al. (1988) J. of Reproduction, 39:511 or PCT application No. PCT/FR93/00598 (WO 94/00568) by Mehtali et al., all of which are hereby incorporated by reference. Preferably, the female is subjected to hormonal conditions effective to promote superovulation prior to obtaining the eggs.

Many techniques can be used to introduce DNA into an egg or other nucleated cell, including in vitro fertilization using sperm as a carrier of exogenous DNA (“sperm-mediated gene transfer”, e.g., Lavitrano et al., 1989, Cell 57: 717-723), microinjection, gene targeting (Thompson et al., 1989, Cell 56: 313-321), electroporation (Lo, 1983, Mol. Cell. Biol. 3: 1803-1814), transfection, or retrovirus mediated gene transfer (Van der Putten et al., 1985, Proc. Natl. Acad. Sci. USA 82: 6148-6152). For a review of such techniques, see Gordon (1989), Transgenic Animals, Intl. Rev. Cytol. 115:171-229. All of their references are hereby incorporated by reference.

Except for sperm-mediated gene transfer, eggs should be fertilized in conjunction with (before, during or after) other transgene transfer techniques. A preferred method for fertilizing eggs is by breeding the female with a fertile male. However, eggs can also be fertilized by in vitro fertilization techniques.

Fertilized, transgene containing eggs can than be transferred to pseudopregnant animals, also termed “foster mother animals,” using suitable techniques. Pseudopregnant animals can be obtained, for example, by placing 40-80 day old female animals, which are more than 8 weeks of age, in cages with infertile males, e.g., vasectomized males. The next morning, females are checked for vaginal plugs. Females who have mated with vasectomized males are held aside until the time of transfer.

Recipient females can be synchronized, e.g. using GNRH agonist (GnRH-a): des-glylo, (D-Ala6)-LH-RH Ethylamide, SigmaChemical Co., St. Louis, Mo. Alternatively, a unilateral pregnancy can be achieved by a brief surgical procedure involving the “peeling” away of the bursa membrane on the left uterine horn. Injected embryos can then be transferred to the left uterine horn via the infundibulum. Potential transgenic founders can typically be identified immediately at birth from the endogenous litter mates. For generating transgenic animals from embryonic stem cells, see e.g., teratocarcinomas and embryonic stem cells, a practical approach, ed. E. J. Robertson, (IRL Press 1987) or in Potter, et. al. Proc. Natl. Acad. Sci. USA 81, 7161 (1984), the teachings of which are incorporated herein by reference.

Founders that express the gene can then bred to establish a transgenic line. Accordingly, founder animals can be bred, inbred, crossbred or outbred to produce colonies of animals of the present invention. Animals comprising multiple transgenes can be generated by crossing different founder animals (e.g. an HIV transgenic animal and a transgenic animal, which expresses human CD4), as well as by introducing multiple transgenes into an egg or embryonic cell as described above. Furthermore, embryos from A-transgenic animals can be stored as frozen embryos, which are thawed and implanted into pseudo-pregnant animals when needed (See e.g. Hirabayashi et al. (1997) Exp Anim 46: 111 and Anzai (1994) Jikken Dobutsu 43: 247).

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail to tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

Transgenic Chickens

Transgenic chickens are made by procedures well known in the art. For example, Salter, et al., Virology 157:236-240 1987; Love, et al, bio/Technology 12:60-63 (1994); Crittendon, et al., J. Reprod. Fert. Suppl. 41:163-171 (1990); Carscience, et al, Development 117:669-675 (1993) the teachings of which are hereby incorporated by reference describe methods of producing transgenic chickens. In particular, transgenic chickens of the invention may be made by incorporating DNA constructs containing proteins of the pentose phosphate pathway into the genome of avian leukosis viruses and the viruses are injected near the blastoderm of fertile eggs prior to incubation. The embryo of a newly laid fertile egg is pluripotent and the injection of avian leukosis viruses near the embryo serves to infect some germ cells.

Transgenic Cows

Transgenic cows are made by procedures well known in the art. A protocol for the production of transgenic cows can be found in Transgenic Animal Technology, A Handbook, 1994, ed. Carl A. Pinkert, Academic Press, Inc., which is hereby incorporated by reference. DNA constructs containing the proteins of the pentose phosphate pathway are introduced into cows using these procedures.

Milk and Eggs

For milk and eggs, the low allergen cows and chickens can be used directly to produce low allergen milk and eggs. Such low allergen milk and eggs can be consumed directly. Alternatively, the low allergen milk and eggs can be used to make low allergen food products such as breads, cakes, pies and the like. Finally, once selected, the low allergen chicken and cow genotypes can be engineered by genetic engineering to make them even less allergenic. Transgenic cows and chickens can be made by procedures well known in the art. Such transgenic cows and chickens can be engineered to overproduce thioredoxin, NTR, glucose-6-phosphate dehydrogenase and other enzymes to increase the reduction of free thiol groups on thiol-containing proteins to make them less allergenic by changing the redox slate of the free thiol groups on proteins.

Shellfish and Fish

For shellfish such as shrimp and mussels and fish, the low allergen genotypes can be selected for direct consumption by consumers with allergies to shellfish and/or fish. Once selected, the low allergen shellfish or fish can be engineered by genetic engineering to make them even less allergenic. Transgenic shellfish can be made by procedures well known in the art. Such transgenic shellfish can be engineered to overproduce thioredoxin, NTR, glucose-6-phosphate dehydrogenase and other enzymes to increase the reduction of free thiol groups on thiol-containing proteins to make them less allergenic by changing the redox state of the free thiol groups on proteins

The invention will be better understood by reference to the following non-limiting examples.

### EXAMPLES

Unless otherwise indicated, all reagents and biochemicals were obtained from sources previously identified (Kobrehel et al., 1992).

### Example 1

Objective

The purpose of the present study was to compare the allergenic potential of proteins from 7 different wheat genotypes using the atopic dog model described by Ermel et al. (1997). Allergenicity of the wheat was assessed by skin testing dogs for differential sensitivity to the isolated wheat fractions.

Material and Methods

Wheatgrain. The grain was from several sources. The California variety, Yecora Rojo, was obtained for University of California, Davis; Ward, a durum wheat, was from K. Khan (North Dakota State University, Fargo, N. Dak.); the “M” lines were provided by Monsanto and stored at −20° C. The other lines were stored at 4° C.

Wheat sensitization of atopic dogs. From the original inbred colony of highly allergic dogs, breeding resulted in 2 litters (7FA, 7FC, 18 pups), some of which were immunized with commercial preparation of wheat grain (1:10 w/v) from Bayer. The allergic response to the preparation was followed systematically over a two-year period. The colony of high IgE-producing atopic dogs was maintained at the Animal Resources Service, University of California, Davis (Ermel et al., 1997). The animals, representing the 7^{th} generation of the colony, were cared for according to the principles in the NIH Guide for the Care and Use of Laboratory Animals. Either six or four of the 4-year-old dogs from the 7^{th} generation litters that had been sensitized to wheat were used in this study as indicated. Other wheat-sensitive dogs had been culled.

Skin tests: Procedures for skin tests to measure the type I hypersensitivity reaction have been described elsewhere (Ermel et al., 1997; Buchanan et al., 1997; del Val et al., 1999). In brief, Evans blue dye 0.5% (0.2 ml/kg) was injected intravenously 5 minutes prior to skin testing. Aliquots of 0.1 ml of the individual extracts were injected intradermally on ventral abdominal skin. The top concentration of allergens in 0.1 ml equivalent to 10 μg was serially diluted in log steps. Skin tests were read blindly by the same experienced observer scoring two perpendicular diameters of each blue spot.

Extraction of the wheat endosperm proteins. Albumin/globulin, gliadin, and glutenin fractions were isolated according to their differential solubility. One gram of grain was ground with a Wiley mill and extracted sequentially with 3 ml of the following solutions: (i) 0.5 M NaCl for albumins/globulins, (ii) 50% (vol/vol) 1-propanol for the gliadins and (iii) 50% (vol/vol) 1-propanol with 1% glacial acetic acid for the glutenins. Samples were extracted using an electrical rotator at 25° C. and then clarified by centrifugation (25,000×g for 10 min at 4° C.). The resulting supernatant solutions were collected. After estimation of protein concentration, each fraction was serially diluted in physiological buffered saline (PBS) and then used for the skin tests.

Protein Assay. Protein concentration was determined by the Bradford method (Bio-Rad) using bovine gamma globulin as standard (Bradford, 1997).

Data Analysis. The data are presented as the logarithm of the lowest protein concentration giving an allergenic response. As the range of concentrations was quite broad, we applied the logarithm of the dose response for statistical analysis. To this end, we used the mean and the standard deviation of the logarithm obtained with the indicated number of dogs tested for the calculations by the complete randomized block design method. The statistical significance of the differences among the wheat lines tested was determined by two-tailed ANOVA F-tests. The null hypothesis—assuming no difference in allergenic response among the different lines—was tested against the alternative hypothesis—assuming a difference among the lines. The two-tailed F-tests were completed at 0.05 level of significance—i.e., a p value<0.05 reflected statistical difference.

Results

The results demonstrate that the albumin/globulin fraction of the wheat lines differ in allergenicity (Table 1). Statistically, the grains fell into three groups (a the weakest, b intermediate and c the strongest). M1070 showed the lowest allergenicity and M1088 and M1085 the strongest. In real terms, allergenicity of the albumins/globulins between the highest and lowest lines differed by a factor of at least 20-X. (Experimental values are given in a footnote to Table 1 and for the other fractions tested in Tables 2 and 3.) The difference between M1070 and M1085/M1088 was significant statistically (p value=0.0006). The other lines were intermediate as indicated in Table 1.

TABLE 1
Skin test response to albumin plus globulin fractions from different wheat lines.

| | Yecora | | | | | | |
|---|---|---|---|---|---|---|---|
| | Rojo | Ward | M1070 | M1085 | M1088 | M1089 | M1103 |
| Allergenicity^{†} | 2.83 | 2.83 | 3.5 | 2.17 | 1.83 | 2.33 | 3.33 |
| S.D. | 1.17 | 0.98 | 1.05 | 1.47 | 2.14 | 1.63 | 1.21 |
| | abc* | abc | a | c | c | bc | ab |

6 dogs sensitized to a commercial preparation of wheat were used. These animals consistently showed a strong response to albumins and globulins. The p value of F-test (Anova) was 0.0006.
^{†}Mean of the logarithm of the lowest amount of protein giving a reaction. The corresponding responsive real numbers (ng protein) left to right were 676, 676, 3162, 148, 68, 214 and 2138.
*The overlapped alphabet among results means that there is no statistical difference, assuming a p value of 0.05.

The dogs also showed a differential allergic response to the gliadin (alcohol-soluble) fraction (Table 2). As with the albumins/globulins, the M1070 line showed the lowest allergenidty (Group A) in the gliadin fraction and M1085 was second to highest (Group B). The two lines differed by a factor of 500 in real terms (see footnote to Table 2). In this case, Yecora Rojo appeared to be the highest by a narrow margin. The differences among these lines were statistically significant (p value=0.0285). Again, the allergenicity of the other lines was intermediate.

TABLE 2
Skin test response to gliadin fraction from different wheat lines.

| | Yecora | | | | | | |
|---|---|---|---|---|---|---|---|
| | Rojo | Ward | M1070 | M1085 | M1088 | M1089 | M1103 |
| Allergenicity^{†} | 0.00 | 1.00 | 3.00 | 0.25 | 0.75 | 1.25 | 1.50 |
| S.D. | 3.46 | 2.45 | 2.16 | 2.5 | 3.2 | 2.5 | 2.08 |
| | b | ab* | a | b | ab | ab | ab |

4 dogs sensitized to a commercial preparation of wheat were used. These animals consistently showed a strong response to gliadins. The p value of F test (Anova) was 0.0285
^{†}Mean of the logarithm of the lowest amount of protein giving a reaction. The corresponding responsive real numbers (ng protein) left to right were 1, 10, 1000, 2, 6, 18 and 32.
*The overlapped alphabet among results means that there is no statistical difference, assuming p value of 0.05. Note that with the gliadins, the different lines separated statistically into 2 rather than 3 groups (c.f. Table 1).

Analysis of the acid-soluble glutenins revealed that, unlike the albumins/globulins or gliadins, there were no significant differences in allergenicity among the cultivars (Table 3). Nonetheless, once again, the allergenicity of M1070 was the lowest by an overall average of 4-X on an absolute basis (see footnote to Table 3).

TABLE 3
Skin test response to glutenin fraction from different wheat lines.

| | Yecora | | | | | | |
|---|---|---|---|---|---|---|---|
| | Rojo | Ward | M1070 | M1085 | M1088 | M1089 | M1103 |
| Allergenicity^{†} | 2.50 | 2.75 | 3.25 | 2.75 | 2.50 | 2.50 | 3.00 |
| S.D. | 1.73 | 1.89 | 0.96 | 1.5 | 1.73 | 1.73 | 1.15 |
| | a* | a | a | a | a | a | a |

Four dogs sensitized to a commercial preparation of wheat were used. These animals consistently showed a strong response to glutenins.
^{†}Mean of the logarithm of the lowest amount of protein giving a reaction. The corresponding responsive real numbers (ng protein) left to right were 316, 562, 1778, 562, 316, 316 and 1000.

Conclusions

The allergenic response to the fractions isolated from the different wheat lines is summarized below.

Allergenicity of albumin/globulin fraction:

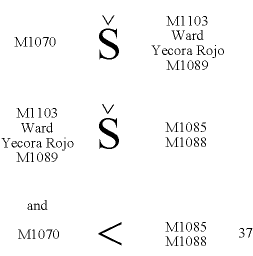

Allergenicity of Gliadin Fraction:

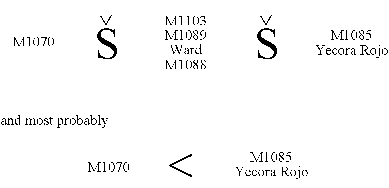

Based on these data, we conclude that M1070 had the lowest allergenicity in both the albumin/globulin and gliadin fractions. M1085 showed very strong allergenidty in both cases. There were other significant differences with M1088 and Yecora Rojo, both of which also showed strong allergenicity, respectively, with the albumins/globulins and gliadins. A comment is in order regarding the relatively high allergenicity of Yecora Rojo. The dogs used in this study have been challenged numerous times in the past with protein fractions of this grain. It is likely, therefore, that during earlier periods, they have developed increased sensitivity to the Yecora Rojo proteins. The dogs were not previously exposed to the other wheat lines tested in this study.

We have tried to determine whether the differences in the mean of the log number of the lowest concentration giving a reaction among the wheat lines could be applied to an authentic population of wheat-sensitive dogs (Table 4). To this end, we calculated the probability of an allergenic response induced within a given line relative to the response of the strongest line. We based the calculation on the lowest amount of protein showing a reaction in 50% of the population responding to the strongest wheat line.

TABLE 4
The probability of different wheat lines to induce an allergenic response with
allergic population of dogs

| | Yecora Rojo | Ward | M1070 | M1085 | M1088 | M1089 | M1103 |
|---|---|---|---|---|---|---|---|
| | % Responding to test concentration | | | | | | |
| Albumin/Globulin | 20 | 15 | 6 | 41 | 50* | 38 | 11 |
| Gliadin | 50* | 34 | 8 | 46 | 41 | 31 | 24 |
| Glutenin | 50* | 45 | 22 | 43 | 50* | 50* | 33 |

*Corresponds to the probability that an allergenic response is induced in 50% of the population of sensitized dogs with the lowest protein concentration found for the strongest allergen. The 50% value (ng protein) was 68 for albumin/globulin (M1088), 148 for gliadin (Yecora Rojo) and 316 for glutenins (Yecora Rojo, M1088 and M1089).

On this basis, with the albumins/globulins, M1070 showed about 40% reduction in allergenicity relative to M1088. In the case of the gliadin fraction, M1070 also showed about 40% reduction compared with Yecora Rojo. The corresponding reduction for M1070 vs. M1085 (the latter showed strong allergenicity), again, was about 40% for both the albumin/globulin and gliadin fractions. The corresponding numbers for the glutenins are also included in Table 4, although they are not statistically significant. Nonetheless, also in this case, M1070 continued to rank lowest in allergenicity relative to the other lines by about 10%.

The above results were used to rank the total allergenicity of the different wheats. In so doing, we ranked the allergenicity of the three protein fractions—albumins/globulins, gliadins, glutenins—for each line. We multiplied the rank number by the relative abundance of the relevant protein fraction: albumin/globulin, 0.2; gliadin, 0.4; and glutenin, 0.4. The product obtained corresponds to the relative allergenicity of the combined fractions. It should be noted that such a ranking assumes no cross-reactivity between fractions. According to this ranking, Yecora rojo and M1088 showed the strongest allergenicity and M1103 and M1070 the lowest (Table 5). It is not possible to relate this ranking quantitatively to the magnitude of total allergenicity of the different lines.

TABLE 5
Ranking of total allergenicity of the different wheat lines. Number 1
corresponds to the most and number 7 to the least allergenic.

| | Ranking of |
|---|---|
| Lines | Total Allergenicity |
| Yecora rojo | 1 |
| M1088 | 2 |
| M1085 | 3 |
| M1089 | 4 |
| Ward | 5 |
| M1103 | 6 |
| M1070 | 7 |

The results show that the protein fractions isolated from grain of 7 different wheat lines differed significantly in allergenicity (by a factor of up to 1,000). Of the 3 fractions analyzed, the water-soluble albumins/globulins showed statistically significant differences (line M1070 was less than either M1085 or M1088). With the ethanol-soluble gliadins, the statistical differences were also significant with M1070 most probably showing less allergenicity than either M1085 or M1088. While there was a trend with the acid-soluble glutenins, there was no statistically significant difference among the lines (line M1070 was still the lowest in allergenicity by a factor of 4). The allergenicity tests were carried out by skin testing wheat-sensitized dogs from a colony of hypersensitive animals. The results demonstrate that the 7 lines of wheat show unexpected differences in allergenicity. Differences were seen in two of the major protein fractions (albumins/globulins and gliadins) that in our hands account for most (about 75%) of the total allergenicity of the grain in dogs as well as humans. Accordingly, the evidence demonstrates that it is prudent to test wheat (or other foods) for allergenicity as it can vary widely among different lines (or sources).

### Example 2

The redox status of the thioredoxin-linked proteins in seeds was investigated in a series of experiments taking advantage of transgenic wheat grains overexpressing thioredoxin h produced using a B-hordein promoter and a signal sequence that targeted the linked protein to the protein body (Cho et al., 1999). Ground grain was extracted sequentially for albumins, globulins, gliadins, and glutenins. The fluorescent probe monobromobimane (mBBr), which preferentially binds to sulfhydryl groups of reduced proteins, was only present in the initial aqueous solvent used for extraction (buffer plus salt). The rationale is that any protein that existed in the sulfhydryl form in the dry grain will be labeled at this step. Two types of analyses were carried out: one in which extracts were labeled without treatment, and a second in which extracts were incubated with two components of the NADP/thioredoxin system—NADPH and NADP-thioredoxin reductase (NTR)—prior to adding mBBr. In this treatment the only thioredoxin h present in the grain is at either the control or overexpressed level. In each of these experiments we compared the proteins that were labeled with mBBr in the homozygous line with those in the corresponding null segregant. Only data on the albumin fraction are being presented in this report.

Materials and Methods

Materials and chemicals. Transgenic wheat (Triticum aestivum L. cv. Yecoro Rojo) lines overexpressing thioredoxin h were generated as previously described for cereals (Cho et al., 1999; Kim et al. 1999). Chlamydomonas reinhardtii thioredoxin h, and Arabidopsis thaliana NTR were kind gifts of J.-P. Jacquot (Université de Nancy I, Vandoeuvre, France).

Chemicals. Reagents for IEF and SDS-polyacrylamide gel electrophoresis were purchased from Bio-Rad Laboratories (Hercules, Calif.). Monobromobimane (mBBr) or Thiolite was obtained from Calbiochem Co. (San Diego, Calif.). Other chemicals and biochemicals were purchased from commercial sources and were of the highest quality available.

Protein Extraction. Wheat grains (3^{rd} generation) from greenhouse-grown plants were ground in a Wiley Mill fitted with a 40-mesh screen. One gram ground wheat grain was extracted with 20 ml 5% NaCl in 20 mM Tris-HCl, pH 7.5 containing 2 mM mBBr at 25° C. for 30 min. Excess mBBr was derivatized with 2-mercaptoethanol. The resultant supernatant fraction was dialyzed against 100-fold excess of the Tris-HCl buffer overnight at 4° C. After centrifugation (15 min at 27,000×g), the supernatant fraction (containing the albumins) was divided into 2-ml aliquots and stored at −80° C. until use.

In vivo and in vitro Reduction of Protein. The control experiments were designed to ascertain the in vivo reduction status of proteins in the ground transgenic grain with no extra treatment. A second treatment was designed to visualize the effect of overexpressed thioredoxin h in the presence of excess reducing power by adding NADPH and NTR. In the latter case the two components were incubated first for 10 min at 37° C., added to the grain extract without mBBr and then incubated for 60 min at 37° C. mBBr was then added, the solution incubated for 15 min, and the sample processed as described above.

Reversed Phase HPLC Chromatography. Thawed aliquots of the albumin extracts from equivalent amounts of homozygote and null segregant grain were clarified by centrifugation (10 min at 14,000 rpm). A two-ml filtered sample was injected into a Sephasil Protein C4 column (5 um ST 4.6/250) that had been equilibrated with Buffer A (H₂O containing 0.1% trifluoroacetic acid or TFA). After washing with 12 ml Buffer A to remove unbound protein, the column was eluted with a gradient of 20% to 80% Buffer B (acetonitrile containing 0.1% TFA) on a BioCad Sprint System (PE Biosystems) equipped with both fluorescent and UV detectors. One-ml fractions were collected. The fractions containing protein were either lyophilized or treated as indicated below.

SDS-Reducing 1D PAGE. mBBr-labeled albumin samples, from the reversed phase step above, that had been previously reduced by thioredoxin h were dissolved in Laemmli sample buffer, and subjected to electrophoresis in 10 to 20% Criterion gel at a constant voltage of 150 on a Criterion Precast Gel System (Bio-Rad). After electrophoresis, the image of fluorescent protein bands was captured using Quantity One on a Gel Doc 1000 (Bio-Rad) over a 365-nm UV light box. The proteins were then stained with 0.025% Coomassie brilliant blue G-250 in 10% acetic acid, and de-stained in the same acetic acid solution without the dye. Protein patterns were captured as above using a white light instead of UV light box. Proteins were quantified using the Volume Tools of Quantity One Quantitation Software, Version 4 (Bio-Rad). The mean value—i.e., the intensity of the pixels inside the volume boundary—was measured for each protein band in question.

IEF/SDS-Reducing 2D PAGE. A 2-ml aliquot of each of the original albumin samples was thawed and clarified extract was desalted and concentrated in Ultrafree-15 Centrifugal Filter Unit with 5,000 MWCO membrane. The concentrated sample was buffer-exchanged with 1-ml rehydration buffer twice. The equilibrated sample was added to IPG strips (pH 5-8), rehydrated for 10 h at 20° C. in rehydration tray on the Protean IEF Cell (Bio-Rad). Isoelectric focusing was performed in a Protean IEF Cell using a preset program with 35,000 total voltage-hour and an upper voltage limit of 8,000 V. After termination of isoelectric focusing, the IPG strip was removed and dipped in Equilibration Tricine buffer for 20 min. Then the strip was applied horizontally to a 16.5% Peptide Criterion gel, and electrophoresis in the second dimension was performed at constant 150 V at 25° C. for 1.5 h on a Criterion Precast Gel System (Bio-Rad). Fluorescent and protein images were captured as described above.

Identification of Protein Targets. Reduction/alkylation and trypsin in-gel digestion of mBBr-labeled proteins were carried out essentially by the procedure described by Shevchenko et al. (1996). Extracted trypsin-digested peptides from gels were separated by microbore C18 reversed-phase column (1 mm×25 cm; Vydac, Hesperia, Calif.) on ABI 172 HPLC system (Applied Biosystems). After injection of the sample, the column was washed with 95% solvent A (0.1% TFA in water), 5% solvent B (0.075% trifluoroacetic acid in 70% acetonitrile) for 5 min for column equilibration. The column was eluted first with a linear gradient from 5% to 10% solvent B for 10 min, second with a linear gradient from 10% to 70% B for 70 min that increased to 90% solvent B over 15 min.

Sequence analysis of C18-purified peptides was performed at the Molecular Structure Facility (University of California, Davis) by automated Edman degradation on an ABI model 494 Procise sequencer (Applied Biosystems). Nontarget proteins were analyzed by nano-electrospray ionization tandem mass spectrometry (nano ESI/MS/MS) using a hybrid mass spectrometer QSTAR (Perkin-Elmer). Nano-spray capillaries were obtained from Protana (Odense, Denmark). For nano ESI/MS/MS, in gel digested peptide mixture was analyzed directly without any C18 column fractionation.

Results

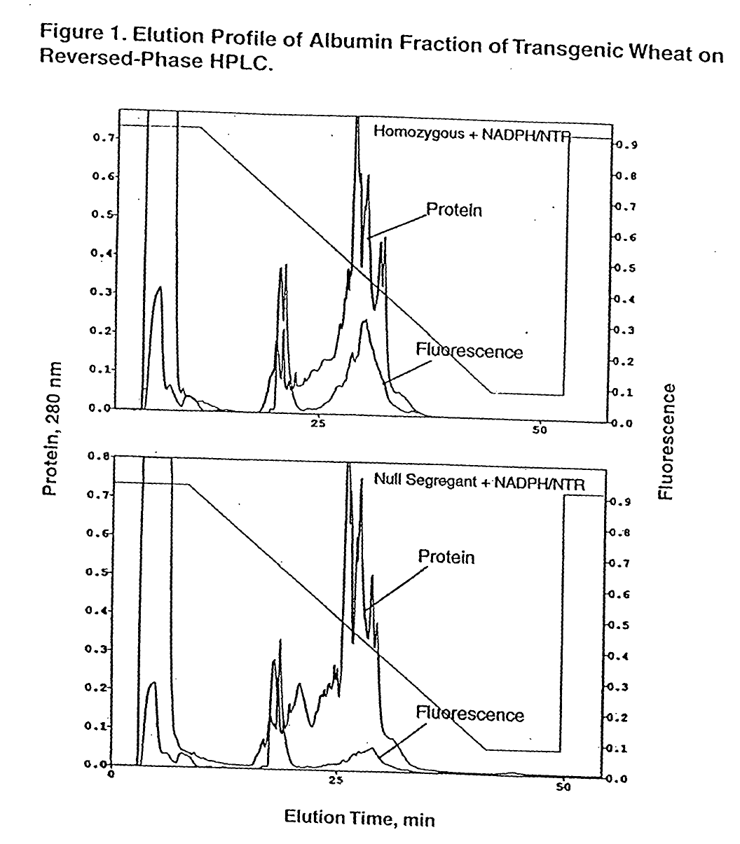

Analyses revealed that there was extensive fluorescent label in the albumin fraction using the above labeling and protein fractionation techniques. The relative reduction of protein (area of fluorescence/protein) was calculated from the elution profile obtained on a C4 reversed phase column. A significant (ca. 11%) difference was noted in the reduction of proteins from the homozygous wheat line overexpressing thioredoxin h relative to the null segregant counterpart (Table 6, Experiment I). Moreover, with added NADPH and NTR, this difference increased to 3.9-fold (Table 6, Experiment II). As there were notable differences in the reversed phase column profiles of the homozygote and the null segregant extracts with NADPH and NTR (FIG. 1), the protein fractions from the two lines were further analyzed by electrophoresis (first 1D SDS-PAGE and then 2D IEF/SDS-PAGE).

TABLE 6
Relative Reduction of Proteins in the Albumin Fraction from a
Homozygous Line of Wheat Overexpressing Thioredoxin h vs.
the Null Segregant either without (Experiment I) or with
Reduction by NADPH and NTR (Experiment II).

| | | Relative | Homozygous/ |
|---|---|---|---|
| Experiment | Line | Reduction* | Null Segregant |
| I. | Homozygous | 0.10519 | 1.11 |
| −NADPH/NTR | | | |
| | Null Segregant | 0.0946 | |
| II. | Homozygous | 0.23211 | 3.91 |
| +NADPH/NTR | | | |
| | Null Segregant | 0.05927 | |

*Area of fluorescence of peaks divided by area of protein of peaks. Area is expressed as micro-Absorbance Units (AU) × sec.

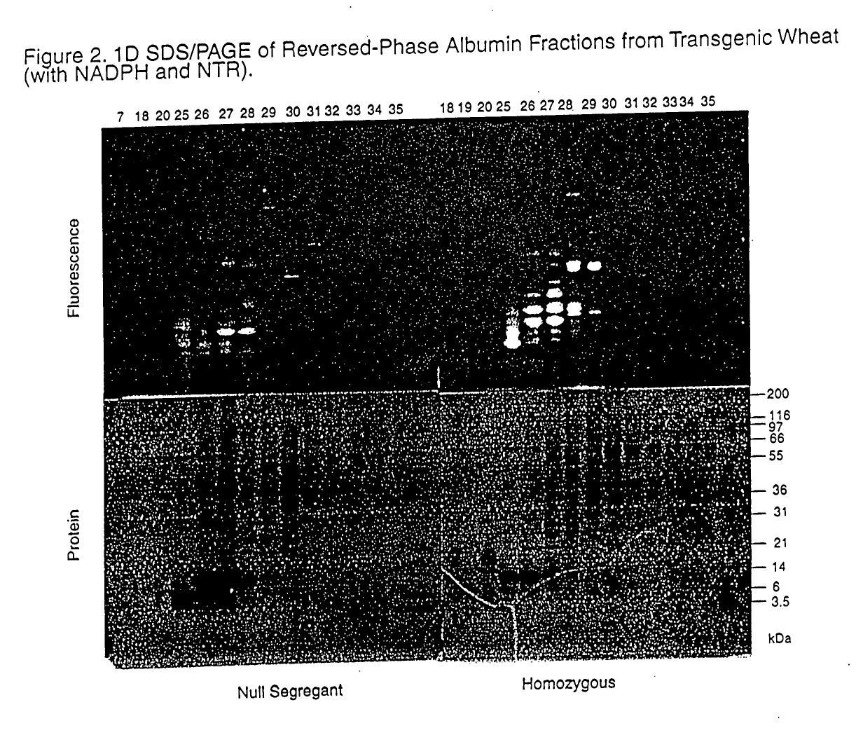

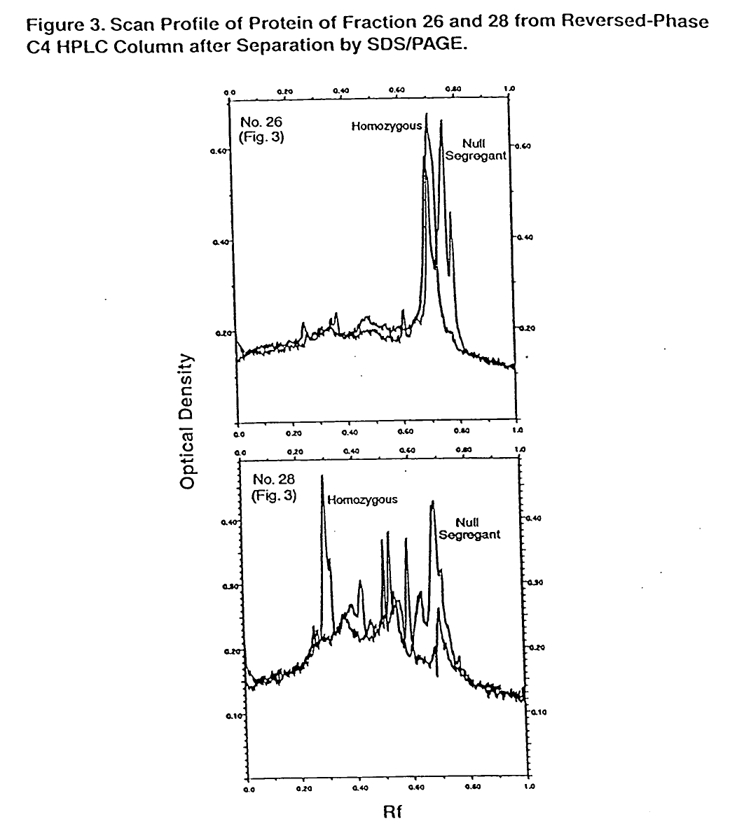

FIG. 2 shows a composite of the fluorescence and protein profiles of selected reversed phase-HPLC fractions of the albumins from the homozygous wheat line overexpressing thioredoxin h (right) and the corresponding null segregant wheat line (left) following treatment with NADPH and NTR. This figure illustrates the upper limit of the proteins that could be reduced in the dry grain of the homozygous wheat line overexpressing thioredoxin h when NADPH and NTR are not limiting. It is interesting to note that the protein patterns from homozygous and null segregant lines were not the same. There seemed to be a decrease in the abundance of protein from the 3.5 to ca.16 kDa region in the homozygote (designated by an asterisk in FIG. 2), particularly an almost complete absence of the band at approximately 3.5 kDa. It is noted that thioredoxin h was detected in fractions 30 to 35 with gel immunoblots (data not shown). Scanning of a 1D SDS-PAGE developed with two of the fractions differing in protein profile from the two wheat lines (nos. 26 and 28) further illustrates the difference in protein pattern eluted from the reversed phase column (FIG. 3). The other fractions analyzed (nos. 25-32) also showed dissimilar protein profiles. In addition to indication of a change in the distribution of proteins in the homozygote, the results presented so far revealed that the albumin fraction contained numerous proteins targeted for reduction by thioredoxin.

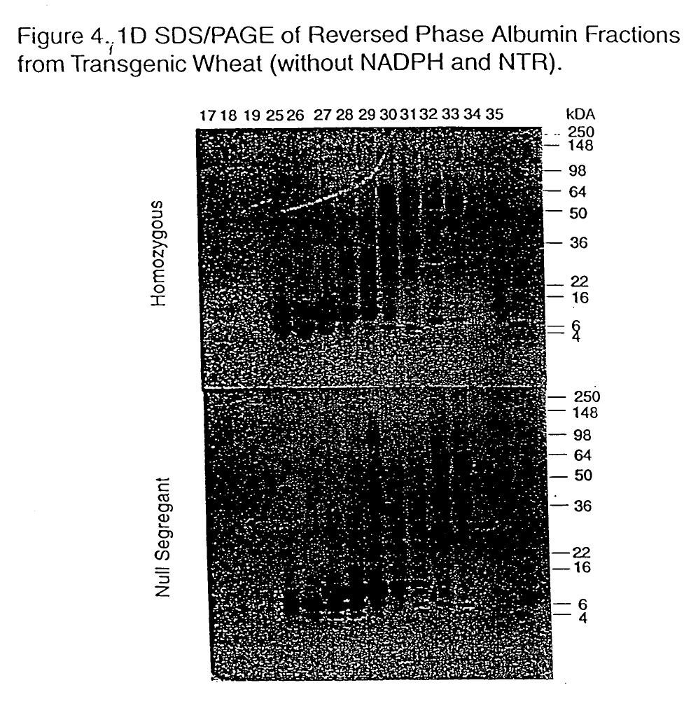

A change in the distribution of albumin proteins was also observed when comparing untreated extracts from the homozygote overexpressing thioredoxin h and the null segregant (FIG. 4). Here we observed a shift in the homozygote similar to that reported above for the NADPH/NTR-treated extracts. Again, especially noteworthy was the general decrease in proteins in the 3.5 to 16 kDa regions in the homozygote and the accompanying absence of the 3.5 kDa band (see asterisk, FIG. 4, top panel). Quantitation revealed that proteins in the 3.5-16 kDa region, that included the alpha-amylase and alpha-amylase/trypsin inhibitors, were reduced by 22% in the homozygote relative to the null segregant (Table 7). On the basis of these results, it appears that the overexpression of thioredoxin effected a change such that the level of certain proteins is decreased. The basis for this change is under investigation.

TABLE 7
Effect of Overexpressing Thioredoxin h on the
Abundance of Albumin Proteins in the 3.5 to 16 kDa Range.
The numbers were obtained with the gels shown in FIG. 4.

| | Mean Optical | Relative |
|---|---|---|
| Grain | Density | Abundance |
| Homozygote | 2,350.0 | 78 |
| Null segregant | 3,007.5 | 100 |

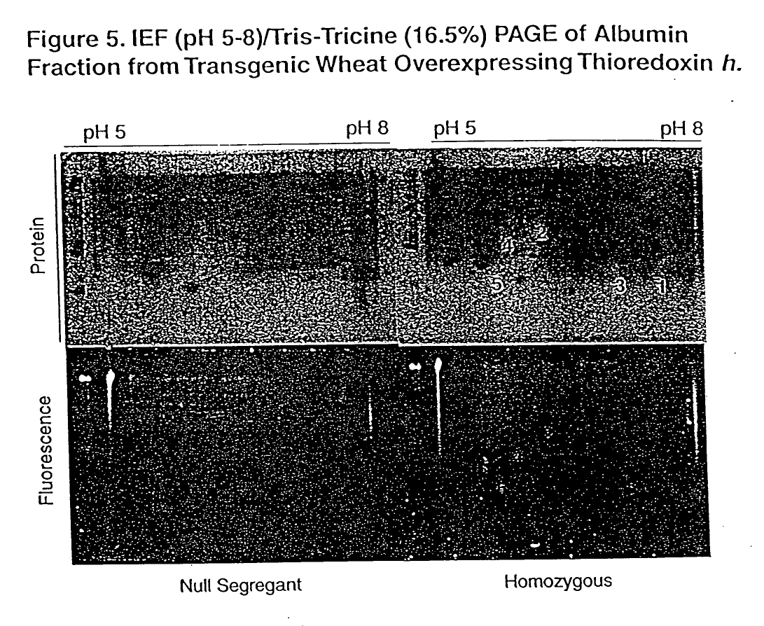

The question arises as to whether, in addition to changing the protein distribution in the albumin fraction, overexpressed thioredoxin h changed their redox state. We have sought an answer to this question by analyzing extracts of the null segregant and homozygote, without treatment with NADPH and NTR, by mBBr/2D IEF/SDS-PAGE (FIG. 5). It may be seen that a number of proteins were more reduced (more fluorescent) in the homozygote. Most of the prominent protein spots were of low molecular mass. When comparing the 2-D gels from the two lines, five proteins were observed to be more highly reduced in extracts of the homozygote (spots 1-5, FIG. 5).

Amino acid sequence analysis led to the identification of three of the purified proteins as wheat alpha-amylase and alpha-amylase/trypsin inhibitors: spot #1 was an alpha-amylase inhibitor isoform with a calculated pI of 6.66, #3 was an alpha-amylase/trypsin inhibitor and #4 was a mixture of an alpha-amylase inhibitor isoform (pI 5.23) plus thioredoxin h (Table 8). Alpha-amylase inhibitors are reported to be the major cause of Baker's asthma (Amano et al., 1998). Significantly, the proteins in spots numbers 1 and 4 showed 100% identity with one of the alpha-amylase inhibitor allergens (0.19 inhibitor) (Maeda et al., 1985) whose allergenic properties were studied by Amano et al. (1998). The alpha-amylase inhibitors identified in this study can thus be considered isoforms of this allergen that show a similar molecular weight but different isoelectric points (FIG. 5). Members of this protein family were earlier found to be reduced by thioredoxin in vitro (Kobrehel et al., 1991), and when so reduced to show loss of activity and increased susceptibility to digestion by trypsin (Jiao et al., 1992; 1993). Based on this property, the alpha-amylase inhibitors of the transgenic grain would be more digestible (hyperdigestible) and less allergenic (hypoallergenic) compared to the null segregant counterpart (del Val et al., 1999 and references therein). The proteins inhibiting trypsin would not only lose activity and be more digestible, but would also be more sensitive to heat and susceptible to proteases (Jiao et al., 1991; 1993). The decreased abundance of the inhibitor proteins would also contribute significantly to lowering the total allergenicity and trypsin inhibitory activity of the homozygous grain. Spot #5 was identified as an isoform of thioredoxin h (Table 8) that differed in molecular mass from its counterpart in spot #4 (FIG. 5).

Protein #2 of Table 8 showed strong homology to oat avenin (also called “seed storage protein”) (Shotwell et al., 1990)—a wheat gliadin homolog. A minor spot adjacent to #2-#2′—that is not obvious in FIG. 5 was also sequenced and shown to contain an isoform of the wheat gliadin homolog identified in spot #2 (data not shown). As with the alpha-amylase inhibitors, the gliadin isoforms showed a similar molecular weight but different isoelectric points. It is noteworthy that gliadins containing disulfide groups, like the one identified in Table 8, are major food allergens in children (Varjonen et al., 1995). Furthermore, the allergenic effect of these proteins is alleviated following reduction by thioredoxin (Buchanan et al., 1997). On this basis, it can be concluded that the increased reduction of the representative gliadins identified in the homozygote would render the grain less allergenic. It is also possible that this increase in reduction could alter gastrointestinal processing so as to make the grain more tolerant for sufferers of coeliac disease where gliadins have been identified as the causative agent (Buchanan et al., 1997; del Val et al., 1999; Howdle and Blair, 1992; Kagnoff et al., 1982).

TABLE 8
Internal Amino Acid Sequence Analysis Of Thioredoxin Target Proteins In
Transgenic Wheat Overexpressing Wheat Thioredoxin h. The SwissPROT accession numbers are:
Spot #1, p01085; #2, q38794; #3, p16851; #4, p01084 (inhibitor), o64394 (thioredoxin); #5,
o64394. note that the alpha-amylase inhibitors showed similar molecular weights but different
isoelectric points of 6.06 and 5.23 (see FIG. 5). by contrast, the thioredoxin h showed a similar
isoelectric point but differed in molecular mass.

| | Internal | Homologous | | Amino Acid | |
|---|---|---|---|---|---|
| No. | Sequence | Protein | MW | Matches | Identity |
| 1 | SGPWMCYPGQAFQVPALPACR | heat alpha-amylase pI 6.66 inhibitor | 13,337 | 21/21 | 100.0 |
| | (SEQ ID NO: 4) | | | | |
| 2 | DALLQQCSPVADMSFLR | Oat avenin, mature protein* | 22,072 | 14/17 | 82.4 |
| | (SEQ ID NO: 3) | | | | |
| 3 | EYVAQQTCGVGIVGS | Wheat alpha-amylase/trypsin | 15,460 | 15/15 | 100.0 |
| | (SEQ ID NO: 2) | inhibitor | | | |
| 4 | DCCQQLADISEWCR | Wheat alpha-amylase pI 5.23 | 13,185 | 13/14 | 92.9 |
| | (SEQ ID NO: 1) | inhibitor | | | |
| | KFPAAVFLK | Wheat thioredoxin h-type | 13,392 | 9/9 | 100.0 |
| | (SEQ ID NO: 21) | | | | |
| 5 | IMAPIFADLAK | Wheat thioredoxin h-type | 13,392 | 11/11 | 100.0 |
| | (SEQ ID NO: 22) | | | | |

*Wheat gliadin counterpart, also called “seed storage protein.”

Conclusions

Thioredoxin h targeted and overexpressed in the protein body of wheat endosperm effected a significant (11%) increase in the reduction of proteins of the albumin fraction (S-S->2 SH). Included were alpha-amylase and alpha-amylase/trypsin inhibitors and gliadins containing disulfide groups.

Members of the alpha-amylase inhibitor, alpha-amylase/trypsin inhibitor and sulfur-rich gliadin families were among the proteins found to be more reduced in the homozygote in vivo.

Based on in vitro studies, increased reduction of the alpha-amylase/trypsin inhibitor would decrease its ability to inhibit trypsin and increase its susceptibility to heat and digestion by trypsin—i.e., make the protein hyperdigestible.

Thioredoxin h overexpressed in wheat endosperm also effected a change in the distribution of proteins in the albumin fraction such that the level of those in the 3.5 to 16 kDa region, including the alpha-amylase and alpha-amylase/trypsin inhibitors, was decreased by 22% in the homozygote vs. the null segregant.

Based on current evidence, a decreased abundance coupled with an increased reduction, would decrease the allergenicity of proteins of the albumin fraction.

The alpha-amylase inhibitors and the gliadins containing disulfide groups are, respectively, the major cause of Bakers' asthma in adults and wheat allergy in children. The above evidence is, therefore, in accord with the conclusion that the homozygote grain overexpressing thioredoxin h is hypoallergenic and hyperdigestible.

More extensive reduction of the albumin proteins was observed in the homozygote when the reducing potential was not limiting—i.e., when the albumin fraction was incubated with NADPH and NTR to reduce indigenous thioredoxin h, that, in turn, reduced the target proteins. This finding suggests that grain engineered to increase the generation of NADPH (e.g., by overexpressing NTR and/or glucose 6-phosphate dehydrogenase) would enhance the reduction of endosperm proteins beyond that observed in the current study.

The homozygote overexpressing thioredoxin h is being studied with respect to technological properties—i.e., allergenicity, digestibility and baking quality.

### Example 3

Objective

The purpose of the present study was to determine the improvement in the allergenicity of proteins from transgenic wheat (Yecora Rojo) with overexpressed thioredoxin h using the atopic dog model described by Ermel et al. (1997). Allergenicity of the transgenic wheat was compared with that of its null segregant component by skin testing dogs for differential sensitivity to the isolated protein fractions.

Material and Methods

Transgenic wheat grain. Transgenic Yecora Rojo wheat grain with overexpressed thioredoxin h was produced as previously for barley (Cho et al., 1999). The homozygote contained about 25-X increase in the protein level of thioredoxin h relative to the null segregant.

Wheat sensitization of atopic dogs. From the original inbred colony of highly allergic dogs, breeding resulted in 2 litters (7FA, 7FC, 18 pups), some of which were immunized with commercial preparation of whole grain bread wheat (1:10 w/v) from Bayer. The allergic response to the preparation was followed systematically over a two-year period. The colony of high IgE-producing atopic dogs was maintained at the Animal Resources Service, University of California, Davis (Ermel et al., 1997). The animals, representing the 7^{th} generation of the colony, were cared for according to the principles in the NIH Guide for the Care and Use of Laboratory Animals. Either six or four of the 4-year-old dogs from the 7^{th} generation litters that had been sensitized to wheat were used in this study as indicated. Other wheat-sensitive dogs had been culled.

Skin tests: Procedures for skin tests to measure the type I hypersensitivity reaction have been described elsewhere (Ermel et al., 1997; Buchanan et al., 1997; del Val et al., 1999). In brief, Evans blue dye 0.5% (0.2 ml/kg) was injected intravenously 5 minutes prior to skin testing. Aliquots of 0.1 ml of the individual extracts were injected intradermally on ventral abdominal skin. The top concentration of allergen in 0.1 ml equivalent to 10 μg was serially diluted in log steps. Skin tests were read blindly by the same experienced observer scoring two perpendicular diameters of each blue spot.

Extraction of the wheat endosperm proteins. Albumin/globulin, gliadin, and glutenin fractions were isolated according to their differential solubility. One gram of grain was ground with a Wiley mill and extracted sequentially for the indicated times with 3 ml of the following solutions: (i) 0.5 M NaCl for albumins/globulins, 30 min (ii) 70% (vol/vol) ethanol for the gliadins, 2 hr and (iii) 0.1M glacial acetic acid for the glutenins, 2 hr. Samples were extracted using an electrical rotator at 25° C. and then clarified by centrifugation (25,000×g for 10 min at 4° C.). The resulting supernatant solutions were collected. After estimation of protein concentration, each fraction was serially diluted in physiological buffered saline (PBS) and then used for the skin tests.

Protein Assay. Protein concentration was determined by the Bradford method (Bio-Rad) using bovine gamma globulin as standard (Bradford, 1997).

Data Analysis. The data are presented as the logarithm of the lowest protein concentration giving an allergenic response. As the range of concentrations was quite broad, we applied the logarithm of the dose response for statistical analysis. To this end, we used the mean and the standard deviation of the logarithm obtained with the indicated number of dogs tested for the calculations by the complete randomized block design method. The statistical significance of the differences between the homozygote and the null segregant was determined by one-tailed sign rank test. The null hypothesis—assuming no difference in allergenic response between the homozygote and the null segregant—was tested against the alternative hypothesis—assuming a difference between two. The one-tailed sign rank tests were completed at 0.05 level of significance—i.e., a p value<0.05 reflected statistical difference.

Results

Table 9 demonstrates that the albumin/globulin and glutenin fractions did not differ significantly in allergenicity between homozygote and null segregant. Only the gliadin fraction showed a statistically significant difference—i.e., homozygote was less allergenic than null segregant (p=0.033). It seems likely, therefore, that the baker's asthma aeroallergen found earlier to be decreased in the transgenic grain was not detected in the present analyses because this protein is a member of the albumin fraction.

TABLE 9
Skin test response to wheat proteins.

| | Albumin | | Gliadin | | Glutenin | |
|---|---|---|---|---|---|---|
| | Null | HZ | Null | HZ | Null | HZ |
| Allergenicity | 2.34 | 2.35 | 3.40 | 3.92 | 2.38 | 2.54 |
| S.D. | 1.10 | 1.54 | 2.72 | 2.27 | 1.32 | 1.42 |

| | | | |
|---|---|---|---|
| Significance | 0.481 | 0.033 | 0.182 |
| (p value) | | | |

Null: null segregant,
HZ: homozygote

Six dogs sensitized to a commercial preparation of wheat were used to test the albumins/globulins. These animals consistently showed a strong response to this fraction. Four dogs were used to test the gliadins and glutenins. Each of these animals displayed consistent sensitivity to these fractions over 2-year period.

† Mean of the logarithm of the lowest amount of protein giving a reaction. The corresponding responsive real numbers (ng protein) left to right were 219, 224, 2512, 8318, 240 and 347.

We have tried to determine whether the differences in the mean of the log number of the lowest concentration giving a reaction between homozygote and the null segregant could be applied to an authentic population of wheat-sensitive dogs (Table 10). To this end, we calculated the probability of an allergenic response induced within a given homozygote relative to the response of the null segregant. We based the calculation on the lowest amount of protein showing a reaction in 50% of the population responding to the null segregant.

TABLE 10
Probability of different proteins of transgenic wheat to induce an
allergenic response with allergic population of dogs.

| | | |
|---|---|---|
| Albumin/globulin | Gliadin | Glutenin |

% Responding to test concentration

| | | | |
|---|---|---|---|
| Null | 50* | 50* | 50* |
| HZ | 50 | 41 | 45 |

allergenic response with allergic population of dogs.
Null: null segregant,
HZ: homozygote
*Corresponds to the probability that an allergenic response is induced in 50% of the population of sensitized dogs with the lowest protein concentration found for the null segregant.

The 50% value (ng protein) was 219 for albumin/globulin, 2512 for gliadin and 240 for glutenins.

On this basis, with the gliadin fraction, the homozygote showed about a 10% reduction in allergenicity relative to the null segregant. The corresponding numbers for the albumins/globulins and the glutenins are also included in Table 10, although they are not statistically significant. Nonetheless, with the glutenins, the homozygote continued to show a trend and was lower in allergenicity than the null segregant by about 5%. In the case of the albumins/globulins, there is no indication of a difference between homozygote and null segregant. Interestingly, these finding are similar to those obtained previously by applying reduced thioredoxin to the isolated Yecora Rojo protein fractions (Buchanan et al., 1997). That is, thioredoxin mitigated the allergenidty of the gliadins and glutenins but not of the albumins or globulins. Testing of additional glutenin-sensitive dogs should show whether or not the glutenin difference is significant.

Conclusions

As determined by skin tests with the dog model, thioredoxin h overexpressed in transgenic grain effected a decrease in the allergenic potential of the gliadin fraction. On the basis of this difference, we calculated a 10% reduction in allergenicity in the gliadin fraction of the homozygous transgenic grain with overexpressed thioredoxin h (homozygote) compared with the null segregant.

### Example 4

Isolation of the Glucose-6-Phosphate Dehydrogenase Gene from Hordeum vulgare

Introduction

There are promising demonstrations of the effects of adding the components of a naturally occurring redox system, NADP/thioredoxin system (NTS), to grains in vitro that lead to the production of value-added grains as well as human and animal nutraceuticals. There are three components to this system: thioredoxin (TRX), NADP thioredoxin reductase (NTR) and NADPH.

Thioredoxins are small ubiquitous proteins (12-14 kDa), that play a variety of physiological roles in the animal, plant and bacterial kingdoms (Holmgren 1985). The protein contains a disulfide bridge between two cysteine residues in the active center, WCGPC (Trp-Cys-Gly-Pro-Cys), which in heterotrophic tissues is reduced by NADP thioredoxin reductase (Holmgren 1985). Higher plants are known to possess two types of thioredoxin systems, ferredoxin/thioredoxin system (FTS) and NTS, and three types of thioredoxins, m, f, and h (Jacquot et al. 1997). The NADP/thioredoxin system (NTS) is analogous to the system in animals and most microorganisms where thioredoxin (h-type in plants) is reduced by NTR and NADPH is used as an electron donor (Johnson et al. 1987a, Florencio et al. 1988, Suske et al. 1979).

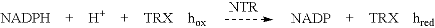

The driving force of the reaction is the source of electrons, NADPH. This coenzyme can be generated through glucose-6-phosphate dehydrogenase (G6DPH), which catalyzes the first step of the oxidative pentose phosphate pathway (OPPP), namely the conversion of glucose-6-phosphate to 6-phosphogluconolactone. Concomitantly, NADPH is generated. The main function of G6PDH is to generate NADPH for anabolic metabolism, including fatty acid synthesis, amino acid, and ribose synthesis (Copeland ant Turner 1987, Turner and Turner 1980, Dennis et al. 1997).

G6PDH has been found in bacteria, yeast and animal tissues as a homodimer or a homotetramer with a subunit size of 50 to 57 kDa (Levy 1979). In plants, at least two isoenzymes have been found, one in the cytosol and one in the plastid with approximately 65% to 75% identity in the amino acid sequences of the two enzymes (Herbert et al. 1979, Srivastava and Anderson 1983). The plastidic G6PDH is regulated by covalent redox modification via the ferredoxin/thioredoxin system (FTS), whereas the regulation of the cytosolic isoform appears to be regulated by the ratio of NADP⁺/NADPH (Fickenscher and Scheibe 1986, Buchanan 1991). The studies of Wenderoth et al. (1997) show that the position of the cysteine residues in the two potato isoenzymes is completely different and that the two cysteine residues (Cys 149 and Cys 157) are involved in the redox regulation of plastidic G6PDH. The complete genomic plastidic clone from tobacco has been isolated and characterized. In addition complete cDNAs have been identified from a number of plant species, including tobacco, Arabidopsis, alfalfa, parsley, wheat and maize (Knight et al. 2001, Fahrendorf et al. 1995, Nemoto and Sasakuma 2000, Redinbaugh and Campbell 1998, Graeve et al. 1994, Batz et al. 1998).

The NTS has been implicated in a wide variety of biological functions. It appears to be involved in developmentally related processes (Brugidou et al. 1993), self-incompatibility (Li et al. 1995) and as a translocation element in sieve tubes (Ishiwatari et al. 1995). In cereals, NTS functions as a signal to enhance metabolic processes during germination and early seed development (Kobrehel et al 1992, Lozano et al. 1996, Besse et al. 1996). Serrato et al. (2001) found two forms of thioredoxin h, which are most abundant in mature seeds. Thioredoxin h also functions in the reduction of intramolecular disulfide bridges of low molecular-weight cysteine-rich proteins, including thionins (Johnson et al. 1987b), protease inhibitors and α-amylase inhibitors (Kobrehel et al. 1991). Moreover, gliadins and glutenins, the major wheat storage proteins, are reduced by NTS (Kobrehel et al. 1992). The addition of NTS to wheat flour was shown to improve dough quality, apparently by reduction of intramolecular disulfide bonds of flour proteins. These bonds then undergo sulfhydryl/disulfide interchanges to form new intermolecular disulfide bonds, thereby contributing to further network formation and stronger doughs (Wong et al. 1993). In addition, it has been shown that reduction by NTS of disulfide protein allergens from wheat and milk in vitro decreased their allergenicity (Buchanan et al. 1997, del Val et al. 1999). The NTS treatment also increases the digestibility of trypsin and α-amylase inhibitors and β-lactoglobulin, a major allergen in milk (del Val et al. 1999). Snake venom neurotoxins are also reported to be reduced and inactivated by NTS (Lozano et al. 1994). A recent study with transgenic barley plants that overexpress wheat TRX h in the endosperm showed that the seed progeny have enhanced activity of a starch-debranching enzyme (pullulanase) in germinating barley seeds (Cho et al. 1999).

These promising demonstrations of the effects of adding the components of NTS in vitro to grains, and in one in vivo case to transgenic grains, open the doors to new avenues to produce value-added grains. In order to utilize genetic engineering approaches to the production of this grain, it is necessary to have the genes for the various components. The barley trx h and ntr genes were cloned and transgenic wheat plants overexpressing TRX h and NTR have been produced and initial studies conducted (unpublished). TRX h and NTR in transgenic wheat grains were expressed at levels 2 to 20 times those of wild type. Now it is of interest to determine the effects of overexpressing another component, the generator of NADPH, that could limit the reactivity of the total NTS. Since a major function of G6DPH is the generation of NADPH, the introduction of the gene encoding this protein should be able to supply additional NADPH and possibly enhance the activity of NTS. The cDNA sequence of barley g6pdh is presented here and the nucleotide and deduced amino acid sequences are compared with known g6pdh sequences from other organisms.

Materials and Methods

Amplification of barley cDNA library. To amplify barley cDNA libraries, the bacterial strain SOLR was streaked on M9 minimal medium including thiamine and grown at 37° C. for 36 hrs. A single colony was chosen and inoculated into LB broth plus 30 mg/ml kanamydn for approximately 4 hrs. An aliquot of the barley cDNA library phagemid stock, unstressed Morex shoots (Hordeum vulgare L. cv. Morex) shoots from 5-day old seedlings grown in the dark was mixed with the bacterial culture and incubated for 15 min at 37° C. After incubation, cells were spread onto LB agar plates containing 30 mg/ml kanamycin and 100 mg/ml ampicillin (to select for the phagemid) and incubated overnight at 37° C. Colonies were collected and phagemids were isolated using a Qiagen plasmid maxi kit (Qiagen, UK).

Identification of partial fragment of barley genomic g6pdh. Two primers were designed based on the cDNA sequence of wheat glucose-6-phosphate dehydrogenase: WG6PD 7 (5′-TACTTGGAAAAGAGTTGGTCCA-3′) (SEQ ID NO: 23) and WG6PD 9R (5′-GATTCCATATTGATCAAAATATCC-3′) (SEQ ID NO: 24). PCR was performed in a programmable thermal controller (MJ Research, Inc, USA). The reaction mixture contained 400 nmol of each primer, 50 μM dNTPs, 40 U/ml pfu DNA polymerase (Staratagene, USA), and 20 μg/ml of barley genomic DNAs (HKK, from selected phagemids?). The PCR product was analyzed using a 0.8% agarose gels. The 450 bp-band was excised and purified using Qiaquick gel extraction kit (Qiagen, UK) and sequenced using an automated sequencer.

Obtaining of the complete cDNA sequence of g6pdh. Based on the partial sequence of the barley genomic g6pdh fragment obtained above, two primers were designed: BG6PD 12R (5′-AGTGGTAAGAACAAACGGTTCGCA-3′) (SEQ ID NO: 25) and BG6PD 13 (5′-CAGATTGTATTCAGGGAGGACT-3′) (SEQ ID NO: 26). These primers, M13F and M13R were combined for PCR reactions for isolated cDNA phagemids as follows: M13F/M13R plus WG6PD 7/BG6PD 13, and M13F/M13R plus WG6PD 9R/BG6PD 12R. PCR products were gel-purified and sequenced. DNA sequence data of all PCR products were combined and the complete cDNA of barley g6pdh was determined.

Results

Identification of partial fragment of barley genomic g6pdh. Using PCR primers, WG6PD 7 and WG6PD 9R, resulted in an amplification product of approximately 500 bp-from barley genomic DNA. The nucleotide sequence of this fragment is highly homologous to the wheat g6pdhs gene. Two more primers were designed based on this fragment, i.e., BG6PD12 and BG6PD13.

Obtaining the complete sequence of the barley g6pdh gene. Combinations of different primers, e.g., either WG6PD 7, WG6PD 9R, BG6PD 12R or BG6PD 13 plus either M13F or M13R, were used to amplify ˜800 to 900-bp fragments from isolated phagemids from the barley cDNA library. The sequences of the overlapping PCR products were combined and the complete g6pdh cDNA sequence was determined. The barley cytosolic cDNA clone has an open reading frame of 509 amino acids. The estimated molecular weight is 57,864 Da and predicted pI is 6.26. The nucleotide sequence of the barley g6pdh gene shows 98% identity with three g6pdhs genes from Triticum aestivum, 88% with Oryza sativa, 77% with Nicotiana tabacum, and 74% with Arabidopsis thaliana. Its deduced amino acid sequence has 96% identity with the three g6pdhs genes of Triticum aestivum, 95% with Oryza sativa, 81% with Nicotiana tabacum, and 78% with Arabidopsis thaliana (FIG. 7).

### Example 5

Transformation of the NTS System

This example illustrates one method of transforming plants with components of the NTS system.

Expression Vector Constructs

Expression vectors are constructed using standard techniques of molecular biology. Once constructed, the vectors may be sequenced prior to transformation to verify that the construct was made correctly. The design of the construct or constructs will depend upon the intended method of introducing multiple genes into the target plant. The expression vectors may be introduced individually or as multigene constructs. Expression vectors introduced individually may be introduced serially into the same plant line. Alternatively, the expression vectors may be introduced into different plant lines. Once stably transformed, the expression vectors may be combined into a single plant line through standard breeding techniques.

Stable Transformation

Transformation of barley, wheat and rice is conducted as previously described above and in Lemaux et al., 1996; Cho et al., 1998; Kim et al., 1999. Trx h alone, ntr alone, G6PDH alone or a mixture of the three genes are used for bombardment with a Bio-Rad PDS-1000 He biolistic device (BioRad, Hercules, Calif.) at 900 or 1100 psi. After obtaining transgenic lines, they may be analyzed to test the redox state, germinability, and allergenicity of the transformed plant.

According to the above examples, other types of plants, are transformed in a similar manner to produce transgenic plants overexpressing thioredoxin and NTR either alone or in combination, such as transgenic wheat, rice, maize, oat, rye sorghum, millet, triticale, forage grass, turf grass, soybeans, lima beans, tomato, potato, soybean, cotton, tobacco etc. Further, it is understood that thioredoxins other than wheat or barley thioredoxin or thioredoxin h can be used in the context of the invention. Such examples include spinach h; chloroplast thioredoxin m and f, bacterial thioredoxins (e.g., E. coli) yeast, and animal and the like. In addition, it is understood the NTR other than barley NTR protein also can be used in the context of the invention such as spinach, wheat, and NTR of monocots and dicots.

This invention has been detailed both by example and by description. It should be apparent that one having ordinary skill in the relevant art would be able to surmise equivalents to the invention as described in the claims which follow but which would be within the spirit of the foregoing description. Those equivalents are included within the scope of this invention. All herein cited patents, patent applications, publications, references and references cited therein are hereby expressly incorporated by reference in their entirety.

### REFERENCES

Amano M, Ogawa H, Kojima K, Kamidaira T, Suetsugu S, Yoshihama M, Satoh, T, Samejima T and Matsumoto I (1998) Identification of the major allergens in wheat flour responsible for Baker's asthma. Biochem J 330:1229-34
Besse I and Buchanan B B 1997 Thioredoxin-linked plant and animal processes: The new generation. Bot Bull Acad Sinica 38: 1-11 Buchanan B B, Adamidi C, Lozano R M, Yee B C, Momma M, Kobrehel K, Ermel R, Frick O L 1997 Thioredoxin-linked mitigation of allergic responses to wheat. Proc Natl Acad Sci USA 94:5372-5377
Besse I et al. (1996) Thiocalsin: a thioredoxin-linked, substrate-specific protease dependent on calcium. Proc Natl Acad Sci USA 93: 3169-3175
Bradford, M. (1976) Anal. Biochem. 72, 248-254.
Brugidou et al. (1993) The Nicotiana tabacum genome encodes two cytoplasmic thioredoxin genes which are differently expressed. Mol Gen Genet 238: 285-293
Buchanan, B. B., Adamidi, C., Lozano, R. M., Yee, B. C., Momma, M., Kobrehel, K., Ermel, R., & Frick, O. L. (1997) Thioredoxin-linked mitigation of allergic responses to wheat. Proc. Natl. Acad. Sci. USA 94:5372-5377.
Buchanan B B (1991) Regulation of CO₂ assimilation in oxygenic photosynthesis: the ferredoxin/thioredoxin system. Arch Biochem Biophys 288: 1-9
Buchanan B B et at (1997) Thioredoxin-linked mitigation of allergic responses to wheat. Proc Natl Acad Sci USA 94: 5372-5377
Calliu M, del Val G, Cho M-J, Lemaux P G and Buchanan B B 1998 Barley gene for thioredoxin. UC Case No. B99-63
Cho M-J et al. (1999) Overexpression of thioredoxin h leads to enhanced activity of starch debranching enzyme (pullulanase) in germinating barley seeds. Proc Natl Acad Sci USA 96: 14641

Cho M-J, Wong J H, Marx C, Wen J, Lemaux P G and Buchanan B B (1999) Overexpression of thioredoxin h leads to enhanced activity of starch debranching enzyme (pullulanase) in barley grain. Proc Natl Acad Sci USA 96:14641-14646
Copeland L and Turner J F (1987) The regulation of glycolysis and the pentose-phosphate pathway. The Biochemistry of Plants 11: 107-125del Val, G., Yee, B. C., Lozano, R. M., Buchanan, B. B., Ermel, R. W., Lee, Y. M. & Frick, O. L. (1999) Thioredoxin treatment increases digestibility and lowers allergenicity of milk J. Allerg. Clin. Immunol. 104: 690-697.
del Val G et al. (1999) Thioredoxin treatment increases digestibility and lowers allergenicity of milk. J Allergy Clinical Immunol 103: 690-697
del Val G, Yee B C, Lozano R M, Buchanan B B, Ermel R W, Lee Y M, Frick O L 1999 Thioredoxin treatment increases digestibility and lowers allergenicity of milk. J Allergy Clin Immunol 103:690-6977

Dennis D T et al. (1997) Glycolysis, the pentose phosphate pathway and anaerobic respiration. Plant Metabolism 105-127
Ermel, R., & Frick, O. L. (1997) Thioredoxin-linked mitigation of allergic responses to wheat. Proc. Natl. Acad. Sci. USA 94:5372-5377.
Ermel, R. W., Kock, M., Griffey, S. M., Reinhart, G. A. & Frick, O. L. (1997) The atopic dog: A model for food allergy. Laboratory Animal Science 47:40-49.
Fahrendorf T et al. (1995) Stress responses in alfalfa (Medicago sativa L.) XIX. Transcriptional activation of oxidative pentose phosphate pathway genes at the onset of the isoflavonoid phytoalexin response. Plant Mol Biol 28: 885-900Ermel, R. W., Kock, M., Griffey, S. M., Reinhart, G. A. & Frick, O. L. (1997) The atopic dog: A model for food allergy. Laboratory Animal Science 47:40-49.
Fickenscher K and Scheibe R (1986) Purification and properties of the cytoplasmic glucose-6-phosphate dehydrogenase from pea leaves. Arch Biochem Biophys 247: 393-402
Florencio F, Yee B C, Johnson T C, and Buchanan B B 1988 An NADP/thioredoxin system in leaves: purification and characterization of NADP-thioredoxin reductase and thioredoxin h from spinach. Arch Biochem Biophys 266:496-507
Florencio F J et al (1988) An NADP/thioredoxin system in green leaves. Arch Biochem Biophys 266: 496-507
Frick, O. L., Gertz, E., Wong, J. H., del Val, G., Yee, B. C., Buchanan, B. B., Petersen, W. R., Teuber, S. S. & Ross, R. (1999). New evidence strenthening the dog as a model for human allergies. Amer. Acad. Allergy Asthma. Immunol. 1999 Annual Meeting, Orlando, Fla. J. Allergy Clin. Immunol. 103: S98.
Graeve K et al. (1994) Purification, characterization, and cDNA sequence of glucose-6-phosphate dehydrogenase from potato (Solanum tuberosum L.). Plant J 5: 353-361
Herbert M et al. (1979) A survey for isoenzymes of glucose phophate isomerase, phosphoglucomutase, glucose 6-phosphate dehydrogenase and 6-phosphogluconate dehydrogenase in C₃, C₄, and Crassulacean-acid-metabolism plants and green algae. Planta 145: 95-104
Holmgren A (1985) Thioredoxin. Ann Rev Biochem 54: 237-271
Howdle P D and Blair G E 1992 Molecular biology and coeliac disease. Gut 33:573-575
Ishiwatari Y et al. (1995) Thioredoxin h is one of the major proteins in rice phloem sap. Planta 195: 456-463
Jacquot J-P et al. (1997) Thioredoxin: structure and function in plant cells. New Phytol 136: 543-570
Johnson T C et al. (1987a) Thioredoxin and NADP-thioredoxin reductase from cultured carrot cells. Planta 171: 321-331
Johnson T C et al. (1987b) Reduction of purothionin by the wheat seed thioredoxin system and potential function as a secondary thiol messanger in redox control. Plant Physiol 85: 446-451
Knight J S et al. (2001) Isolation and characterization of a full-length genomic clone encoding a plastidic glucose-6-phosphate dehydrogenase from Nicotiana tabacum. Planta 212: 499
Kobrehel K et al. (1991) Role of the NADP-thioredoxin system in the reduction of α-amylase and trypsin inhibitor proteins. J Biol Chem 266: 16135-16140
Kobrehel K et al. (1992) Specific reduction of wheat storage proteins by thioredoxin h. Plant Physiol 99: 919-924
Levy R (1979) Glucose-6-phosphate dehydrogenases. Adv Enzymol 48: 97-192
Li X et al. (1995) Thioredoxin activity in the C. terminus of Phalaris S protein. Plant J 8: 133-138
Lozano R M et al. (1994) Thioredoxin-linked reductive inactivation of venom neurotoxins. Arch Biochem Biophy 309: 356-362
Lozano R M et al (1996) New evidence for a role for thioredoxin h in germination and seedling development. Planta 200: 100-106
Nemoto Y and Sasakuma T (2000) Specific expression of glucose-6-phosphate dehydrogenase (G6PDH) gene by salt stress in wheat (Triticum aestivum L.). Plant Sci 158: 53-60
Redinbaugh M G and Campbell W H (1998) Nitrate regulation of the oxidative pentose phosphate pathway in maize (Zea mays L.) root plastids: induction of 6-phophogluconate dehydrogenase activity, protein and transcript levels. Plant Sci 134: 129-140
Jiao J, Yee B C, Kobrehel K and Buchanan B B 1992 Effect of thioredoxin-linked reduction on the activity and stability of the Kunitz and Bowman-Birk soybean trypsin inhibitor proteins. J Agric Food Chem 40:2333-2336
Jiao J, Yee B C, Wong J H, Kobrehel K and Buchanan B B 1993 Thioredoxin-linked changes in regulatory properties of barley alpha-amylase/subtilisin inhibitor protein. Plant Physiol Biochem 31:799-804
Johnson T C, Wada K, Buchanan B B and Holmgren A 1987 Purothionin: reduction by the wheat seed thioredoxin system and potential function as a secondary thiol messenger in redox control. Plant Physiol 85:446-451
Kagnoff M F, Austin R K, Johnson, H C, Bernardin J E, Dietler M D and Kasarda, D D 1982 Celiac sprue: correlation with murine T cell responses to wheat gliadin components. J Immunol 129:2693-2697
Kim H K, Lemaux P G, Buchanan B B and Cho M-J 1999 Reduction of genotype limitation in wheat (Triticum aestivum L.) transformation. Congress on In Vitro Biol 35:43-A
Kobrehel K, Yee B C and Buchanan B B 1991 Role of the NADP thioredoxin system in the reduction of alpha-amylase and trypsin inhibitor proteins. J Biol Chem 266: 16135-16140
Lozano R M, Wong J H, Yee B C, Peters A, Kobrehel K, and Buchanan B B 1996 New evidence for a role for thioredoxin h in germination and seedling development. Planta 200:100-106
Maeda K, Wakabayashi S and Matsubara H 1985 Complete amino acid sequence of alpha-amylase inhibitor in wheat kernel (0.19-inhibitor). Biochim Biophys Acta 828:213-221
Marcus F, Chamberlain S H, Chu C, Masiarz F R, Shin S1 Yee B C and Buchanan B B 1991 Plant thioredoxin h: an animal-like thioredoxin occurring in multiple cell compartments. Arch Biochem Biophys 287:195-198
Serrato A J et al. (2001) Characterization of two thioredoxins h with predominant localization in the nucleus of aleurone and scutellum cell of germinating wheat seeds. Plant Mol Biol 46: 361-371
Shevchenko A, Wilm M, Vorm O and Mann M 1996 Mass spectrometric sequencing of proteins from silver-stained polyacrylamide gels. Anal Chem 68:850-858
Shotwell M A, Boyer S K, Chesnut R S and Larkins B A 1990 Analysis of seed storage protein genes of oats. J Biol Chem 265:9652-9658
Srivastava D K and Anderson L E (1983) Isolation and characterization of light- and dithiothreitol-modulatable glucose-6-phosphate dehydrogenase from pea chloroplasts. Biochem Biophys Acta 724: 359-369
Suske G et al. (1979) NADPH thioredoxin reductase and a new thioredoxin from wheat. Z Naturforsch C 34: 214-221
Turner J F and Turner D H (1980) The regulation of glycolysis and the pentose phosphate pathway. The Biochemistry of Plants 2: 279-316
Varjonen E, Vainio E, Kalimo K, Juntunen-Backman K, Savolainen J 1995 Skin-prick test and RAST responses to cereals in children with atopic dermatitis. Characterization of IgE-binding components in wheat and oats by an immunoblotting method. Clin Exp Allergy 25:1100-1107
Wenderoth I et al. (1997) Identification of the cysteine residues involved in redox modification of plant plastidic glucose-6-phosphate dehydrogenase. J Biol Chem 272: 26985-26990
Wong J H et al. (1993) Thioredoxin and bread wheat. Cereal Chem 70: 113-114
Yano H, Wong J H, Lee Y M, Cho M-J and Buchanan B B 2001 A strategy for the identification of proteins targeted by thioredoxin. Proc Natl Acad Sci USA 98:47944799Batz O et al (1998) Extensive reprogramming of primary and secondary metabolism by fungal elicitor or infection in parsley cells. J. Biol Chem 379: 1127-1135

We claim:

1. A method for selecting a genotype within a species that induces a reduced allergic reaction in an allergy test compared to other genotypes within the species, comprising:
a) testing said genotypes for an allergic reaction in an allergy test and
b) selecting a genotype within said species that exhibits a reduced allergic reaction compared to other genotypes within said species in said allergy test.

2. The method of claim 1 wherein said species is a plant or animal species.

3. The method of claim 2 wherein said plant is selected from the group consisting of wheat, barley, corn, rice, soybean, peanut, Brazil nut, English walnut, kiwis, citrus trees, oak and birch.

4. The method of claim 3 wherein said animal is selected from cows, chickens, shellfish and fish.

5. The method of claim 1, further comprising the step of
c) genetically modifying said selected genotype to further reduce the allergic reaction inducing response of said genotype.

6. The method of claim 5 wherein said genotype is a plant genotype.

7. The method of claim 6 wherein said plant is plant is genetically modified by plant breeding techniques.

8. The method of claim 6 wherein said plant is genetically modified by genetic engineering techniques.

9. The method of claim 1 wherein said allergy test is selected from the group consisting of skin prick test, skin injection test, oral challenge test, blood test, gastroendoscopy test, inhalation test, and transdermal patch test.

10. A method for selecting a genotype within a species that induces a reduced allergic reaction in an allergy test compared to other genotypes within the species, comprising:
a) isolating protein fractions from said genotypes;
b) testing said protein fractions for an allergic reaction in an allergy test; and
c) selecting a genotype within said species that exhibits a reduced allergic reaction compared to other genotypes within said species in said allergy test.

11. The method of claim 10 wherein said species is a plant or animal species.

12. The method of claim 11 wherein said plant is selected from the group consisting of wheat, barley, corn, rice, soybean, peanut, Brazil nut, English walnut, kiwis, citrus trees, oak and birch.

13. The method of claim 12 wherein said animal is selected from cows, chickens, shellfish and fish.

14. The method of claim 10, further comprising the step of
d) genetically modifying said selected genotype to further reduce the allergic reaction inducing response of said genotype.

15. The method of claim 14 wherein said genotype is a plant genotype.

16. The method of claim 15 wherein said plant is plant is genetically modified by plant breeding techniques.

17. The method of claim 15 wherein said plant is genetically modified by genetic engineering techniques.

18. The method of claim 10 wherein said allergy test is selected from the group consisting of skin prick test, skin injection test, oral challenge test, blood test, gastroendoscopy test, inhalation test, and transdermal patch test.

19. A method for selecting a subgroup within a species that induces a reduced allergic reaction in an allergy test compared to other members within the species, comprising:
a) isolating protein fractions from said members;
b) testing said protein fractions for an allergic reaction in an allergy test and
c) selecting a subgroup within said species that exhibits a reduced allergic reaction compared to other members within said species in said allergy test.

20. The method of claim 19 wherein the subgroup and members are genotypes.

21. The method of claim 19 wherein the subgroup and members are subspecies.

22. The method of claim 19 wherein said species is a plant or animal species.

23. The method of claim 22 wherein said plant is selected from the group consisting of wheat, barley, corn, rice, soybean, peanut, Brazil nut, English walnut, kiwis, citrus trees, oak and birch.

24. The method of claim 23 wherein said animal is selected from cows, chickens, shellfish and fish.

25. The method of claim 19, further comprising the step of
d) genetically modifying said selected genotype to further reduce the allergic reaction inducing response of said genotype.

26. The method of claim 25 wherein said genotype is a plant genotype.

27. The method of claim 26 wherein said plant is plant is genetically modified by plant breeding techniques.

28. The method of claim 26 wherein said plant is genetically modified by genetic engineering techniques.

29. The method of claim 19 wherein said allergy test is selected from the group consisting of skin prick test, skin injection test, oral challenge test, blood test, gastroendoscopy test, inhalation test, and transdermal patch test.

30. A recombinant nucleotide comprising SEQ ID NO 20.

31. A recombinant nucleotide comprising a sequence coding for the protein SEQ ID NO 9.

32. An isolated polypeptide comprising SEQ ID NO 9.

33. A vector comprising the nucleotide of claim 30 or 31 operably linked to a promoter sequence.

34. A transgenic plant comprising the vector of claim 33.

35. A transgenic plant comprising:
a) recombinantly expressed thioredoxin h;
b) recombinantly expressed glucose 6 phosphate dehydrogenase; and
c) recombinantly expressed NADP-thioredoxin reductase.


SEQUENCE LISTING (26 sequences)

SEQ ID NO: 1 - protein, 14 aa, Triticum aestivum
DCCQQLADISEWCR

SEQ ID NO: 2 - protein, 15 aa, Triticum aestivum
EYVAQQTCGVGIVGS

SEQ ID NO: 3 - protein, 17 aa, Triticum aestivum
DALLQQCSPVADMSFLR

SEQ ID NO: 4 - protein, 21 aa, Triticum aestivum
SGPWMCYPGQAFQVPALPACR

SEQ ID NO: 5 - protein, 331 aa, Hordeum vulgare
MEGSAAAPLRTRVCIIGSGPAAHTAAIYAARAELKPVLFEGWMANDIAAGGQLTTTTDVENFPGFPTGIMGIDLMDNCRAQSVRFGTNILSETVTEVDFSARPFRVTSDSTTVLADTVVVATGAVARRLHFSGSDTYWNRGISACAVCDGAAPIFRNKPIAVIGGGDSAMEEGNFLTKYGSQVYIIHRRNTFRASKIMQARALSNPKIQVVWDSEVVEAYGGAGGGPLAGVKVKNLVTGEVSDLQVSGLFFAIGHEPATKFLNGQLELHADGYVATKPGSTHTSVEGVFAAGDVQDKKYRQAITAAGSGCMAALDAEHYLQEVGAQVGKSD

SEQ ID NO: 6 - protein, 331 aa, Triticum aestivum
MEEAAAGPLHTRVCIIGSGPAAHTAAVYAARAELKPVLFEGWLANDIAAGGQLTTTTDVENFPGFPDGILGIDLMDRCRAQSVRFGTKIFSETVTSVDFSSRPFRVSSDDTVVHADSVVVATGAVARRLHFAGSDAFWNRGITACAVCDGAAPIFRNKPIAVVGGGDSAMEEANFLTKYGSRVYIIHRRDAFRASKIMQARALSNPKIQVVWDSEVVEAYGGSDGGPLGGVKVKNLVTGEVSDFRVAGLFFAIGHEPATKFLAGQLELDSEGYVATKPGSTHTSVKGVFAAGDVQDKKYRQAITAAGSGCMAALDAEHYLQEVGAQEGKTD

SEQ ID NO: 7 - protein, 333 aa, Arabidopsis thaliana
MNGLETHNTRLCIVGSGPAAHTAAIYAARAELKPLLFEGWMANDIAPGGQLTTTTDVENFPGFPEGILGVELTDKFRKQSERFGTTIFTETVTKVDFSSKPFKLFTDSKAILADAVILATGAVAKRLSFVGSGEASGGFWNRGISACAVCDGAAPIFRNKPLAVIGGGDSAMEEANFLTKYGSKVYIIHRRDAFRASKIMQQRALSNPKIDVIWNSSVVEAYGDGERDVLGGLKVKNVVTGDVSDLKVSGLFFAIGHEPATKFLDGGVELDSDGYVVTKPGTTQTSVPGVFAAGDVQDKKYRQAITAAGTGCMAALDAEHYLQEIGSQQGKSD

SEQ ID NO: 8 - protein, 321 aa, Escherichia coli
MGTTKHSKLLILGSGPAGYTAAVYAARANLQPVLITGMEKGGQLTTTTEVENWPGDPNDLTGPLLMERMHEHATKFETEIIFDHINKVDLQNRPFRLNGDNGEYTCDALIIATGASARYLGLPSEEAFKGRGVSACATCDGFFYRNQKVAVIGGGNTAVEEALYLSNIASEVHLIHRRDGFRAEKILIKRLMDKVENGNIILHTNRTLEEVTGDQMGVTGVRLRDTQNSDNIESLDVAGLFVAIGHSPNTAIFEGQLELENGYIKVQSGIHGNATQTSIPGVFAAGDVMDHIYRQAITSAGTGCMAALDAERYLDGLADAK

SEQ ID NO: 9 - protein, 509 aa, Hordeum vulgare
MAGTDSSASSRQSSFNSLAKDLELPLEQGCLTIVVLGASGALPRRKRSRHFYHLFEQGFLQSGEVHIVGYARTNLSDDGLRGRIRAYLKGASEEHVSEFLQLIKYVSGSYDSGEGFEKLNKEISDYEMSNNSGSSRRLFYLALPPSVYPSVCKMIRTYCMSPTSRTGWTRVIVEKPFGRDLDSAEELSSQLGELFQEDQLYRIDHYLGKELVQNLLVLRFANRLFLPLWNRDNVDNIQIVFREDFGTDGRGGYFDQYGIIRDIIQNHLLQVFCLVAMEKPVSLKPEHIRDEKVKVLQSVNPIKDEEVVLGQYQGYKDDPTVPDDSNTPTFASIVLRVHNERWEGVPFILKAGKALNSRKAEIRVQFKDVPGDIFKCKKQGRNEFVIRLQPSEAMYMKLTVKKPGLEMATEQSELDLSYGMRYQDVKIPEAYERLILDTIRGDQQHFVRRDELKAAWQIFTPLLHNIDAGKLKAVSYKPGSRGPKEADELSEKVGYMQTHGYIWIPPTLA

SEQ ID NO: 10 - protein, 509 aa, Triticum aestivum
MAGTDSSASSRQSSFNSLAKDLELPLEKGCLTIVVLGASGDLAKKKTFPALYHLFEQGFLQSGEVHIVGYARTNLSDDGLRGRIRAYLKGASEEHVSEFLQLIKYVSGSYDSGEGFEKLNKEISDYEMSNNSGSSRRLFYLALPPSVYPSVCKMIRTYCMSPTSRAGWTRVIVEKPFGRGLDSAEELSSQLGELFEEDQLYRIDHYLGKELVQNLLVLRFANRLFLPLWNRDNVDNIQIVFREDFGTDGRGGYFDQYGIIRGIIQNHLLQVFCLVAMEKPVSLKPEHIRDEKVKVLQSVNPIKDEEVVLGQYQGYKEDPTVPDDSNTPTFASIVLRVHNERWEGVPFILKAGKALNSRKAEIRVQFKDVPGDIFKCKKQGRNEFVIRLQPSEAMYMKLTVKKPGLEMATEQSELDLSYGMRYQDVKIPEAYERLILDTIRGDQQHFVRRDELKAAWQIFTPLLHDIDAGKLKAVSYKPGSRGPKEADELSEKVGYMQTHGYIWIPPTLA

SEQ ID NO: 11 - protein, 505 aa, Oryza sativa
MSGGSSPRSRRSSFNSLSRDLELPSEQGCLSVIVLGASGDLAKKKTFPALFHLFAQGFIQSGEVHIFGYARSNLSDDGLRERIRGYLKGASEEHLSDFLQHIKYVSGSYDSGEGFEKLNKEISEYEKSNKSESPRRLFYLALPPSVYPSVCKMIRTYCMNPSGWTRVIVEKPFGKDLDSSEELSAQLGELFDENQLYRIDHYLGKELVQNLLVLRFANRLFLPLWNRDNIDNIQIVFREDFGTDGRGGYFDQYGIIRDIIQNHLLQVFCLVAMEKPVSLKPEHIRDEKVKVLQSVNPIKHDEVVLGQYEGYKDDPTVPDDSNTPTFASVVFRVHNERWEGVPFILKAGKALSSRKAEVRVQFKDVPGDIFKCKRQGRNEFVIRLQPSEAMYMKLTVKKPGLEMATEQSELDLSYGMRYQNVKIPEACERLILDTIRGDQQHFVRRDELKAAWQIFTPLLHDIDEGKVKSIPYQPGSRGPKEADELSERVGYMQTHGYIWIPPTLA

SEQ ID NO: 12 - protein, 511 aa, Nicotiana tobaccum
MAASWCIEKRGSIRLDSFRDNDNIPETGCLSIIVLGASGDLAKKKTFPALFNLYRQGFLQSNEVHIFGYARTKISDDDLRGRIRGYLSQGKENEEEVSEFLQLIKYVSGSYDSGEGFSLLDKAIAEHEIAKNSTEGSSRRLFYFALPPSVYPSVCRMIKNYCMNKSDLGGWTRIVVEKPFGKDLASAEQLSSQIGELFDEPQIYRIDHYLGKELVQNLLVLRFANRFFLPLWNRDNIDNIQIVFREDFGTEGRCGYFDEYGIIRDIIQNQLLQVLCLVAMEKPVSQKPEHIRDEKVKVLQSMLPIKDEEVVLGQYEGYKDDPTVPDNSNTPTFATMVLRIHNERWEGVPFIMKAGKALNSRKAEIRVQFKDVPGDIFRCKKQGRNEFVIRLQPSEAMYMKLTVKKPGLEMSTVQSELDLSYRQRYQGVVIPEAYERLILDTIRGDQQHFVRRDELKAAWEIFTPLLHRIDDGEVKPIPYKPGSRGPAEADELLQNVGYVQTHGYICIPPTL

SEQ ID NO: 13 - protein, 515 aa, Arabidopsis thaliana [sequence omitted]

SEQ ID NO: 14 - protein, 122 aa, Hordeum vulgare
MAASATAAAVAAEVISVHSLEQWTMQIEEANTAKKLVVIDFTASWCGPCRIMAPVFADLAKKFPNAVFLKVDVDELKPIAEQFSVEAMPTFLFMKEGDVKDRVVGAIKEELTAKVGLHAAAQ

SEQ ID NO: 15 - protein, 125 aa, Triticum aestivum
MAASAATATAAAVGAGEVISVHSLEQWTMQIEEANAAKKLVVIDFTASWCGPCRIMAPIFADLAKKFPAAVFLKVDVDELKSIAEQFSVEAMPTFLFMKEGDVKDRVVGAIKEELTNKVGLHAAQ

SEQ ID NO: 16 - protein, 122 aa, Oryza sativa
MAAEEGVVIACHNKDEFDAQMTKAKEAGKVVIIDFTASWCGPCRFIAPVFAEYAKKFPGAVFLKVDVDELKEVAEKYNVEAMPTFLFIKDGAEADKVVGARKDDLQNTIVKHVGATAASASA

SEQ ID NO: 17 - protein, 126 aa, Nicotiana tobaccum
MAANDATSSEEGQVFGCHKVEEWNEYFKKGVETKKLVVVDFTASWCGPCRFIAPILADIAKKMPHVIFLKVDVDELKTVSAEWSVEAMPTFVFIKDGKEVDRVVGAKKEELQQTIVKHAAPATVTA

SEQ ID NO: 18 - protein, 114 aa, Arabidopsis thaliana
MASEEGQVIACHTVETWNEQLQKANESKTLVVVDFTASWCGPCRFIAPFFADLAKKLPNVLFLKVDTDELKSVASDWAIQAMPTFMFLKEGKILDKVVGAKKDELQSTIAKHLA

SEQ ID NO: 19 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 20 - DNA, 1530 bp, Hordeum vulgare
atggcgggaactgactcctcggcgtcatcgagacaaagcagttttaactcattagcaaaggatctagaacttcctttggagcaagggtgcctgactatcgttgtacttggggcttctggagaccttgccaagaagaaaacgttcccggcactctaccacctttttgaacaggggttcttacaatctggtgaagtgcatatagttgggtatgcgagaacaaatctttctgatgatgggttgagagggcgcatccgtgcataccttaaaggagcctcagaggagcatgtttcagaattcttgcaattgataaaatatgtcagtggctcctatgacagtggagaaggttttgaaaaactgaacaaggaaatatcagattatgagatgtcaaacaactcaggaagctcccgtaggctcttttacttggcattgcctccatctgtctacccttcagtgtgcaaaatgatccgaacatattgcatgagtccaacttctcgcactggatggactagagtaattgttgagaagccctttggaagggacctggactcagcagaagaattaagttcccaacttggggagctattccaggaagatcaactctacaggattgaccactacttgggaaaagagttggtccaaaacttgcttgtgcttcgttttgcgaaccgtttgttcttaccactttggaaccgtgataatgttgataatatacagattgtattcagggaggacttcggaactgatgggcgtggaggatattttgatcaatatggaatcatccgtgatatcattcagaaccatttgttgcaggttttctgtttggttgcaatggaaaagcctgtatctcttaagcctgagcacattagagatgagaaagtcaaggttctgcaatctgtgaacccgataaaggacgaagaggtagtccttggacaatatcagggctacaaggatgaccctacagtgccagatgactctaataccccaacgtttgcatctattgtacttagggtacacaatgaaagatgggaaggtgtccctttcattcttaaagctggtaaagcattaaactcaagaaaagcagaaattcgtgtgcagttcaaggatgttcccggtgacatttttaaatgtaagaagcaaggaagaaatgagtttgtcatacgcctccagccatcagaagccatgtatatgaaactaactgtgaagaaacctggattggaaatggctactgaacagagtgaacttgatctgtcatatgggatgcgttaccaagatgtcaaaattccagaggcatacgagcgcctcattttggatacaataagaggagaccagcaacactttgtgcgccgggatgagctgaaggctgcctggcagatcttcactcccttgttgcacaacatcgacgctggcaagctgaaggctgtttcatacaagcctggcagccgtggccccaaggaagctgatgaactgagtgagaaggttgggtacatgcagacccacggttacatctggataccacccacccttgcatag

SEQ ID NO: 21 - protein, 9 aa, Triticum aestivum
KFPAAVFLK

SEQ ID NO: 22 - protein, 11 aa, Triticum aestivum
IMAPIFADLAK

SEQ ID NO: 23 - DNA, 22 bp, Artificial Sequence
  Primer
tacttggaaaagagttggtcca

SEQ ID NO: 24 - DNA, 24 bp, Artificial Sequence
  Primer
gattccatattgatcaaaatatcc

SEQ ID NO: 25 - DNA, 24 bp, Artificial Sequence
  Primer
agtggtaagaacaaacggttcgca

SEQ ID NO: 26 - DNA, 22 bp, Artificial Sequence
  Primer
cagattgtattcagggaggact